(19) United States
(12) Patent Application Publication
(10) Pub. No.: US 2025/0169841 A1
(43) Pub. Date: May 29, 2025

(54) GRIPPING FORCEPS AND SUTURING METHOD

(71) Applicants: OLYMPUS CORPORATION, Tokyo (JP); National Cancer Center, Tokyo (JP)

(72) Inventors: Arimasa SUGIMOTO, Hachioji-shi (JP); Satoru NONAKA, Tokyo (JP)

(21) Appl. No.: 19/037,342

(22) Filed: Jan. 27, 2025

Related U.S. Application Data
(63) Continuation of application No. PCT/JP2022/033849, filed on Sep. 9, 2022.

(51) Int. Cl. A61B 17/29

(52) U.S. Cl. CPC A61B 17/29

(57) ABSTRACT

Gripping forceps according to the present invention include a sheath which extends in a longitudinal direction; and a gripping portion which has a rod provided at a distal end of the sheath and extending in the longitudinal direction, a first jaw coupled to a proximal end side of the rod and being opened and closed toward a distal end side of the rod, and a second jaw coupled to the proximal end side of the rod on a side opposite to the first jaw with the rod sandwiched therebetween and being opened and closed toward the distal end side of the rod. The rod has a distal end portion which includes a first convex portion protruding to a first protrusion side toward the first jaw.

### BACKGROUND OF THE INVENTION

### Field of the Invention

The present invention relates to gripping forceps and a suturing method. This application is a continuation application based on International Patent Application No. PCT/JP2022/033849 filed on Sep. 9, 2022, and the content of the PCT international application is incorporated herein by reference.

### Description of Related Art

In recent years, medical staplers have become known as treatment tools for suturing the digestive tract or the like. An operation for suturing the digestive tract or the like is facilitated by using an appropriate medical stapler so that an operation time can be drastically shortened. A medical stapler is used together with an endoscope, for example, in which a treatment target is sutured while being gripped by forceps.

The forceps described in Patent Document 1 are known as forceps for gripping a treatment target. In the gripping forceps, a gripping portion provided on a distal end side includes a rod and two forceps pieces (jaws), and a surgeon can ligate a resection part or the like after treatment by manipulating two manipulation portions (handles) to open and close the two forceps pieces.

Incidentally, a medical stapler performs suturing after full-thickness resection of the stomach wall (tissue) including a mucous membrane layer and a muscle layer which is formed on the abdominal cavity side (outward side of the stomach) from the mucous membrane layer inside the stomach (inward side of the stomach) has been performed and a side edge of a defective part after resection has been drawn into the stomach. When full-thickness resection is performed inside the stomach, air inside the stomach escapes to the abdominal cavity side, which causes contraction of the stomach in shape. For this reason, the approaching direction of the medical stapler toward a defective part in this state of the stomach is likely to be limited to a direction nearly parallel to the surface of the stomach wall.

Patent Document 1: Specification of U.S. Pat. No. 9,603,614

### SUMMARY OF THE INVENTION

However, in the forceps described in Patent Document 1, while a side edge of a defective part after treatment is gripped and pulled to the medical stapler side, when an approaching direction of the medical stapler is a direction nearly parallel to a surface of the stomach wall (tissue), it is difficult to reliably grip the mucous membrane layer and the muscle layer. For this reason, suturing of a defective part by the medical stapler is insufficient with the forceps described in Patent Document 1.

The present invention has been made in consideration of such circumstances, and an object thereof is to provide gripping forceps capable of reliably gripping a mucous membrane layer and a muscle layer at a circumferential edge of a resection hole, and a suturing method for suturing the resection hole using the gripping forceps.

In order to resolve the foregoing problems, this invention provides the following means.

Gripping forceps according to a first aspect of the present invention includes a sheath which extends in a longitudinal direction; and a gripping portion which has a rod provided at a distal end of the sheath and extending in the longitudinal direction, a first jaw coupled to a proximal end side of the rod and being opened and closed toward a distal end side of the rod, and a second jaw coupled to the proximal end side of the rod on a side opposite to the first jaw with the rod sandwiched therebetween and being opened and closed toward the distal end side of the rod. The rod has a distal end portion which includes a first convex portion protruding to a first protrusion side toward the first jaw.

According to the gripping forceps and the suturing method of the present invention, a mucous membrane layer and a muscle layer at a circumferential edge of a resection hole can be reliably gripped, and the resection hole can be sutured using the gripping forceps.

### BRIEF DESCRIPTION OF THE DRAWINGS

FIG. 1 is a view showing an overall constitution of a medical system used for gripping forceps and a suturing method according to a first embodiment of the present invention.

FIG. 2 is a perspective view of a medical stapler of the same medical system.

FIG. 3 is a front view of a cap of the same medical stapler.

FIG. 4 is a perspective view of the same medical stapler in which a stapler gripping portion of the same medical stapler is in a closed state.

FIG. 5 is a front view of the same medical stapler in which the stapler gripping portion of the same medical stapler is in the closed state.

FIG. 6 is a perspective view of the same medical stapler in which the stapler gripping portion of the same medical stapler is in an open state.

FIG. 7 is a front view of the same medical stapler in which the stapler gripping portion of the same medical stapler is in the open state.

FIG. 8 is a side view of the same medical stapler in which the stapler gripping portion of the same medical stapler is in the closed state.

FIG. 9 is a side view of the same medical stapler in which the stapler gripping portion of the same medical stapler is in the open state.

FIG. 10 is a cross-sectional view of the gripping portion including a staple discharge portion.

FIG. 11 is a cross-sectional view of the gripping portion in which a discharge manipulation wire is dragged.

FIG. 12 is an overall view showing the gripping forceps used in an endoscope of the same medical system.

FIG. 13 is a perspective view showing a state in which the medical stapler and the gripping forceps are attached to the same endoscope.

FIG. 14 is a side view showing a state in which a first forceps piece and a second forceps piece of forceps pieces of the same gripping forceps are in the open state with respect to a rod.

FIG. 15 is a side view showing a modification example of the rod of the same gripping forceps.

FIG. 16 is a side view showing a state in which the second forceps piece of the forceps pieces of the same gripping forceps is in the open state with respect to the rod and the first forceps piece is in the closed state.

FIG. 17 is a side view showing a state in which the first forceps piece of the forceps pieces of the same gripping forceps is in the open state with respect to the rod and the second forceps piece is in the closed state.

FIG. 18 is a side view showing a state in which the first forceps piece and the second forceps piece of the forceps pieces of the same gripping forceps are in the closed state with respect to the rod.

FIG. 19 is a view showing a state in which the endoscope is brought close to a lesion in the suturing method using the same gripping forceps according to the first embodiment of the present invention.

FIG. 20 is an explanatory view of a disposing step in the same suturing method.

FIG. 21 is an explanatory view of an inserting step in the same suturing method.

FIG. 22 is an explanatory view of a first opening/closing step in the same suturing method.

FIG. 23 is an explanatory view of a thrusting step in the same suturing method.

FIG. 24 is an explanatory view of a pulling step in the same suturing method.

FIG. 25 is an explanatory view of a first gripping step in the same suturing method.

FIG. 26 is an explanatory view of a second opening/closing step in the same suturing method.

FIG. 27 is an explanatory view of a second gripping step in the same suturing method.

FIG. 28 is an explanatory view of a drawing step in the same suturing method.

FIG. 29 is an explanatory view of a suturing step in the same suturing method.

FIG. 30 is another explanatory view of the suturing step in the same suturing method.

FIG. 31 is another explanatory view of the suturing step in the same suturing method.

FIG. 32 is an enlarged view of a distal end side of gripping forceps according to a second embodiment of the present invention.

FIG. 33 is a side view showing a state in which a first forceps piece of the same gripping forceps is in the open state and the second forceps piece is in the closed state.

FIG. 34 is an enlarged view of the distal end side of gripping forceps according to a third embodiment of the present invention.

FIG. 35 is a side view showing a state in which a first forceps piece and a second forceps piece of the forceps pieces of the same gripping forceps are in the closed state with respect to a rod.

FIG. 36 is an explanatory view of another modification example of gripping forceps according to the present invention.

FIG. 37 is an explanatory view of another modification example of gripping forceps according to the present invention.

FIG. 38 is an explanatory view of another modification example of gripping forceps according to the present invention.

### DETAILED DESCRIPTION OF THE INVENTION

### First Embodiment

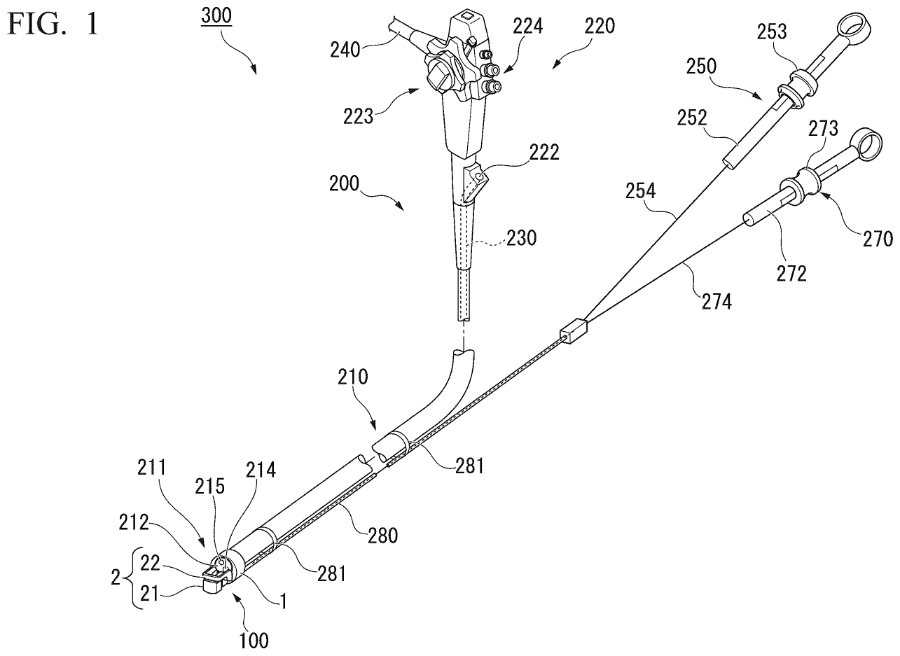

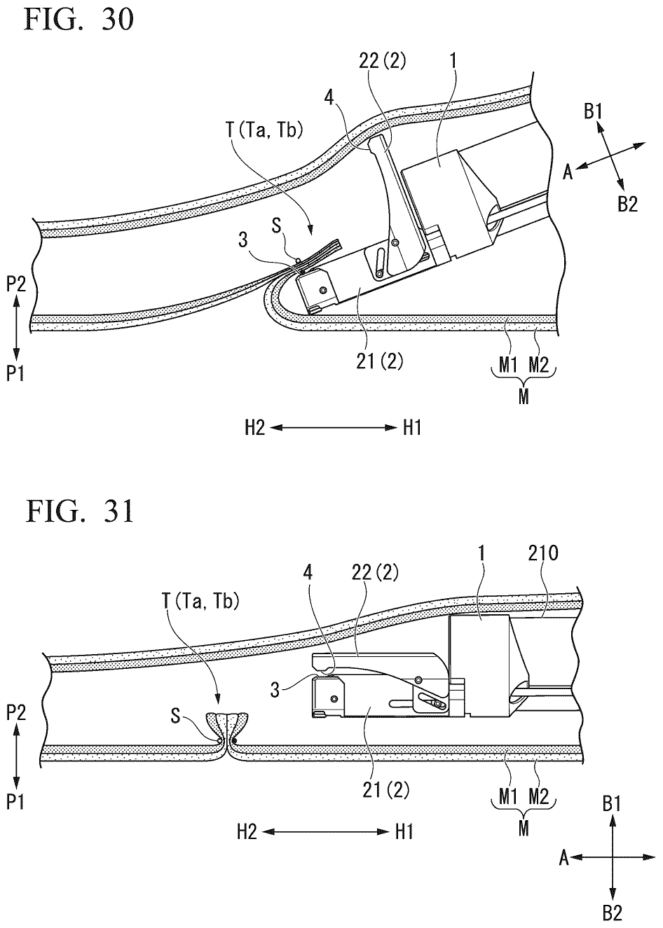

A first embodiment of the present invention will be described with reference to FIGS. 1 to 31.

FIG. 1 is a view showing an overall constitution of a medical system 300 used for gripping forceps (endoscopic treatment tool) 400 and a suturing method according to the present embodiment. The medical system used in the suturing method according to the present embodiment is not limited to the medical system 300.

### [Medical System 300]

The medical system 300 is used in an operation or the like for suturing the digestive tract or the like. The medical system 300 includes a medical stapler 100, an endoscope 200, an opening/closing manipulation portion 250, a discharge manipulation portion 270, and a wire sheath 280. The opening/closing manipulation portion 250 is a manipulation portion for actuating the medical stapler 100 using an opening/closing manipulation wire 254. The discharge manipulation portion 270 is a manipulation portion for actuating the medical stapler 100 using a discharge manipulation wire 274.

### [Endoscope 200]

The endoscope 200 is a known flexible endoscope, which includes a long insertion portion 210 inserted into the body from a distal end, a manipulation portion 220 provided in a proximal end portion of the insertion portion 210, and a universal cord 240.

A treatment tool channel 230 through which the gripping forceps 400 are inserted is formed in the insertion portion 210. A forceps port 214 that is a distal end opening of the treatment tool channel 230 is provided at a distal end 212 of the insertion portion 210. The treatment tool channel 230 extends from the distal end 212 of the insertion portion 210 to the manipulation portion 220.

A distal end portion 211 of the insertion portion 210 includes an image capturing unit (not shown) having a CCD or the like. An objective lens 215 of the image capturing unit is exposed at the distal end 212 of the insertion portion 210.

A knob 223 for manipulating the insertion portion 210 and a switch 224 for manipulating the image capturing unit and the like are provided on a proximal end side of the manipulation portion 220. A surgeon (not shown) can bend the insertion portion 210 in a desired direction by manipulating the knob 223.

A forceps insertion port 222 communicating with the treatment tool channel 230 is provided on a distal end side of the manipulation portion 220. The surgeon can insert the gripping forceps 400 into the treatment tool channel 230 from the forceps insertion port 222.

The universal cord 240 connects the manipulation portion 220 to external peripherals. For example, the universal cord 240 outputs images captured by the image capturing unit to external instruments. The images captured by the image capturing unit are displayed in a display device such as a liquid crystal display via an image processing device.

### [Opening/Closing Manipulation Portion 250]

The opening/closing manipulation portion 250 is a manipulation portion for opening and closing the medical stapler 100 by manipulating the opening/closing manipulation wire 254. As shown in FIG. 1, the opening/closing manipulation portion 250 has an opening/closing manipulation portion main body 252 and an opening/closing manipulation slider 253. A proximal end of the opening/closing manipulation wire 254 is coupled to the opening/closing manipulation slider 253. The surgeon can cause the opening/closing manipulation wire 254 to advance and retract by causing the opening/closing manipulation slider 253 to advance and retract in a longitudinal axis direction with respect to the opening/closing manipulation portion main body 252.

### [Discharge Manipulation Portion 270]

The discharge manipulation portion 270 is a manipulation portion for discharging (ejecting) staples S from the medical stapler 100 by manipulating the discharge manipulation wire 274. As shown in FIG. 1, the discharge manipulation portion 270 has a discharge manipulation portion main body 272 and a discharge manipulation slider 273. The proximal end of the discharge manipulation wire 274 is coupled to the discharge manipulation slider 273. The surgeon can cause the discharge manipulation wire 274 to advance and retract by causing the discharge manipulation slider 273 to advance and retract in the longitudinal axis direction with respect to the discharge manipulation portion main body 272.

### [Wire Sheath 280]

The wire sheath 280 is a sheath through which the opening/closing manipulation wire 254 and the discharge manipulation wire 274 are inserted. As shown in FIG. 1, the distal end side of the wire sheath 280 is coupled to the insertion portion 210 of the endoscope 200 using a band 281.

### [Medical Stapler 100]

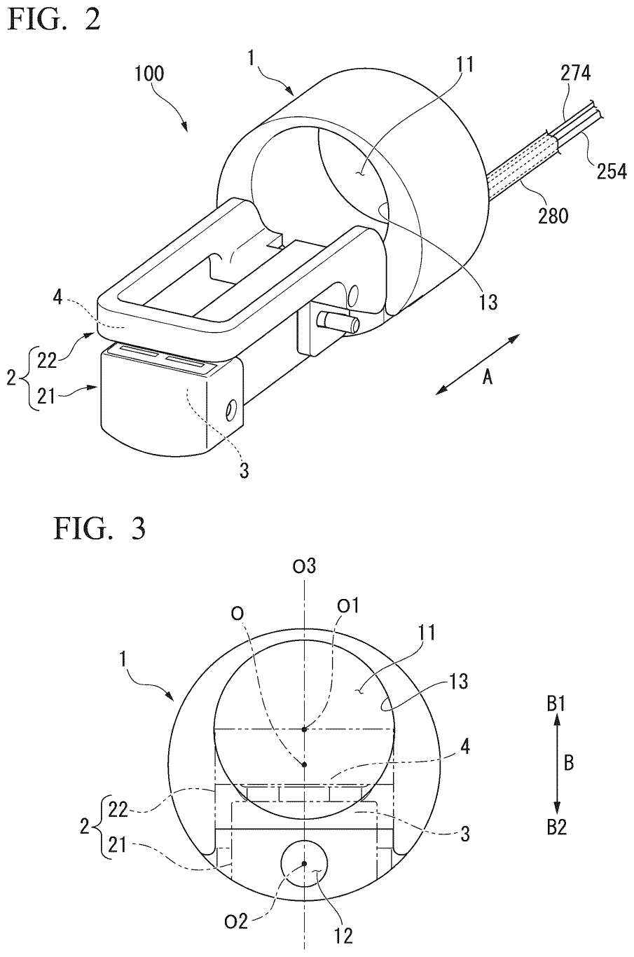

FIG. 2 is a perspective view of the medical stapler 100.

The medical stapler 100 includes a cap (mounting member) 1, a stapler gripping portion 2, a staple discharge portion 3, a staple accommodation portion 4, the opening/closing manipulation wire 254, and the discharge manipulation wire (power transmission member) 274. The medical stapler 100 can be attached and detached with respect to the distal end portion 211 of the insertion portion 210 shown in FIG. 1.

FIG. 3 is a front view of the cap 1 of the medical stapler 100. In FIG. 3, the stapler gripping portion 2 is displayed in a see-through manner.

The cap (mounting member) 1 is a member which can be mounted in the distal end portion 211 of the endoscope 200. The cap 1 is formed to have a substantially columnar shape and has a first penetration hole 11 penetrating it in an axial direction A (FIG. 2) of the medical stapler 100, and a second penetration hole 12 penetrating it in the axial direction A.

The first penetration hole 11 is a hole into which the distal end portion 211 of the insertion portion 210 shown in FIG. 1 is inserted. The shape of the first penetration hole 11 is formed along the external shape of the distal end portion 211 of the insertion portion 210. For this reason, the cap 1 can be mounted in the distal end portion 211 of the endoscope 200 by inserting the distal end portion 211 of the endoscope 200 into the first penetration hole 11.

As shown in FIG. 3, a central axis O1 of the first penetration hole 11 in the axial direction A is eccentric with respect to a central axis O of the cap 1 in the axial direction A. A side in a direction in which the central axis O1 is eccentric with respect to the central axis O will be referred to as “an upward side B1”.

The second penetration hole 12 is a hole into which the wire sheath 280 having the opening/closing manipulation wire 254 and the discharge manipulation wire 274 shown in FIG. 1 inserted therethrough is inserted. The inner diameter of the second penetration hole 12 substantially coincides with the outer diameter of the wire sheath 280.

A distal end portion of the wire sheath 280 is inserted through the second penetration hole 12 and is fixed thereby. The opening/closing manipulation wire 254 and the discharge manipulation wire 274 inserted through the wire sheath 280 pass through the second penetration hole 12 and extend to the distal end side.

As shown in FIG. 3, a central axis O2 of the second penetration hole 12 in the axial direction A is eccentric with respect to the central axis O of the cap 1 in the axial direction A. A side in a direction in which the central axis O2 is eccentric with respect to the central axis O is opposite to the side in the direction in which the central axis O1 is eccentric with respect to the central axis O (upward side B1). A side in a direction in which the central axis O2 is eccentric with respect to the central axis O will be referred to as “a downward side B2”. In the present embodiment, the upward side B1 and the downward side B2 are sides in a vertical direction B.

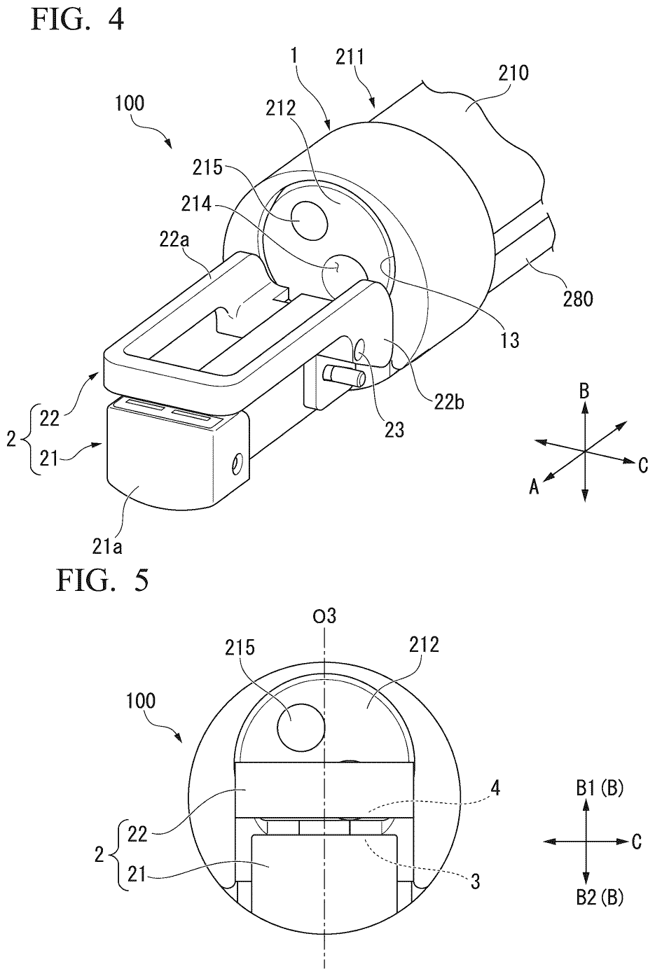

FIG. 4 is a perspective view of the medical stapler 100 in which the stapler gripping portion 2 is in a closed state. FIG. 5 is a front view of the medical stapler 100 in which the stapler gripping portion 2 is in the closed state.

If the cap 1 is mounted in the distal end portion 211 of the endoscope 200, as shown in FIGS. 4 and 5, the objective lens 215 and the forceps port 214 are exposed through an opening 13 of the first penetration hole 11 of the cap 1 on the distal end side. The surgeon can observe a treatment target through the objective lens 215 even in a state in which the medical stapler 100 is mounted in the distal end portion 211 of the endoscope 200.

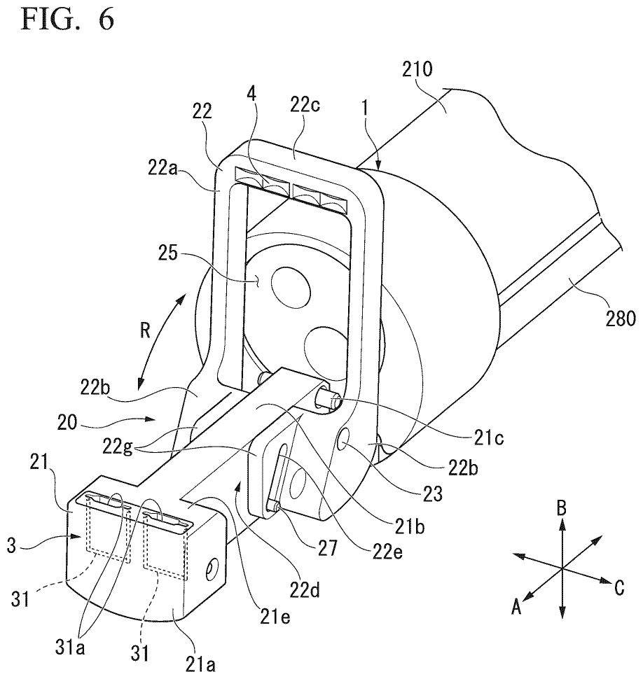

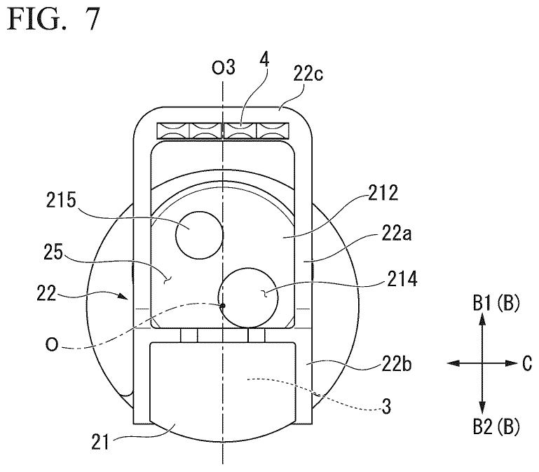

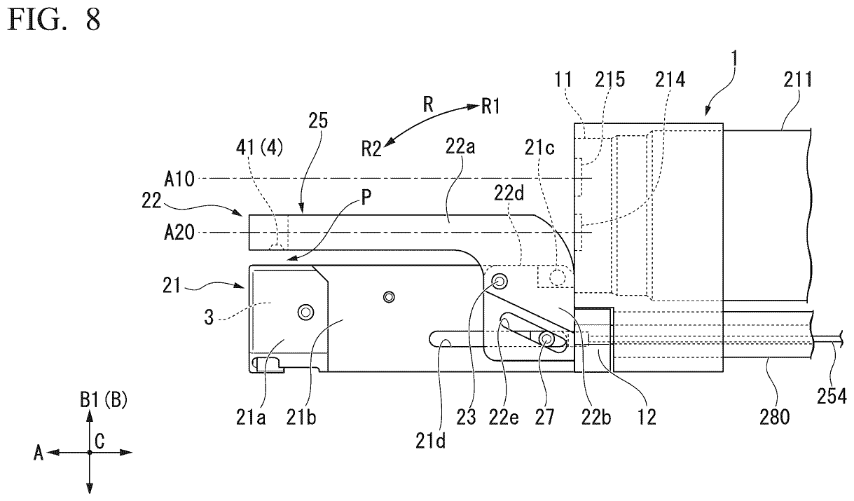

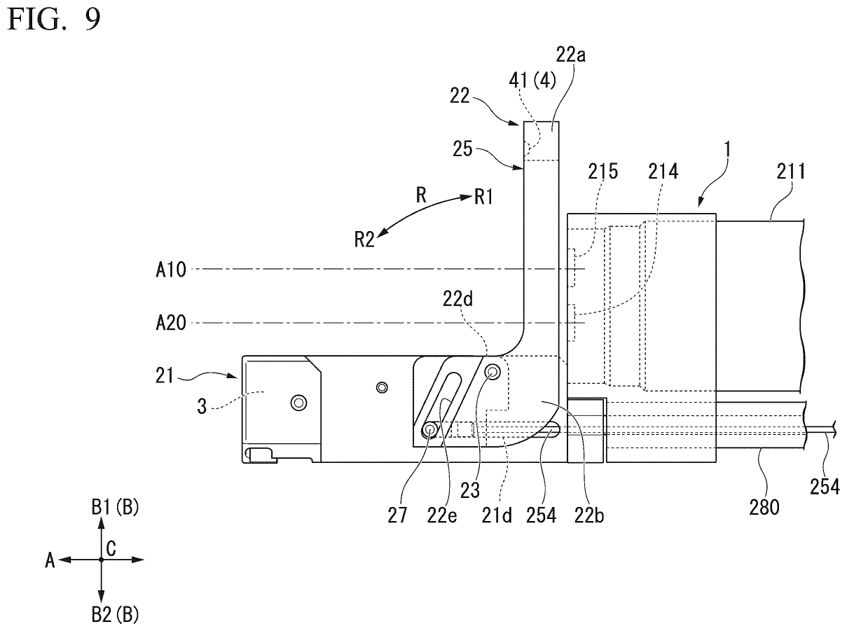

FIG. 6 is a perspective view of the medical stapler 100 in which the stapler gripping portion 2 is in an open state. FIG. 7 is a front view of the medical stapler 100 in which the stapler gripping portion 2 is in the open state. Moreover, FIG. 8 is a side view of the medical stapler 100 in which the stapler gripping portion 2 is in the closed state. FIG. 9 is a side view of the medical stapler 100 in which the stapler gripping portion 2 is in the open state.

As shown in FIG. 6, the stapler gripping portion 2 has a first stapler gripping member 21, a second stapler gripping member 22, an opening/closing rotation shaft 23, and a movable pin 27.

As shown in FIG. 6, the first stapler gripping member 21 and the second stapler gripping member 22 are coupled so as to be openable and closeable by the opening/closing rotation shaft 23. The opening/closing rotation shaft 23 is provided on the distal end side from the cap 1. An axial direction C of the opening/closing rotation shaft 23 is perpendicular to the axial direction A and the vertical direction B of the cap 1. As shown in FIG. 7, the stapler gripping portion 2 is formed symmetrically with respect to a central axis O3 of the vertical direction B.

The first stapler gripping member 21 is fixed to the distal end side of the cap 1 in a non-rotatable manner. The first stapler gripping member 21 is fixed to the cap 1 on the downward side B2 from the central axis O of the cap 1. As shown in FIG. 3, the first stapler gripping member 21 is disposed at a position overlapping the second penetration hole 12 of the cap 1 in a front view. Meanwhile, as shown in FIG. 7, the first stapler gripping member 21 is disposed at a position not overlapping the objective lens 215 and the forceps port 214 of the endoscope 200 in a front view.

As shown in FIG. 6, the first stapler gripping member 21 has a first distal end portion 21a and a first main body portion 21b and is formed to have a substantially T-shape in a plan view. The first distal end portion 21a is disposed on the distal end side from the first main body portion 21b.

The first distal end portion 21a is formed to have a substantially parallelepiped shape. The first distal end portion 21a is formed to have a rectangular shape extending in the axial direction C of the opening/closing rotation shaft 23 in a plan view. The staple discharge portion 3 is provided in the first distal end portion 21a. Openings 31a of the staple discharge portion 3 are provided on a surface (upper surface 21e) of the first distal end portion 21a on the upward side B1.

The first main body portion 21b is a slender member extending in the axial direction A. The distal end of the first main body portion 21b is fixed to the first distal end portion 21a. The proximal end of the first main body portion 21b is fixed to the cap 1 with the wire sheath 280 sandwiched therebetween. The first main body portion 21b has an abutment pin 21c and a first engagement groove 21d (FIG. 8).

The abutment pin 21c is provided at the proximal end of the first main body portion 21b, abuts the second stapler gripping member 22 in the closed state, and regulates a movable range of the second stapler gripping member 22.

As shown in FIG. 8, the first engagement groove 21d is a groove penetrating the first main body portion 21b in the axial direction C of the opening/closing rotation shaft 23. The first engagement groove 21d extends in the axial direction A.

The second stapler gripping member 22 is attached to the first stapler gripping member 21 so as to be rotatable in an opening/closing direction R by the opening/closing rotation shaft 23. As shown in FIGS. 6 and 7, the second stapler gripping member 22 has a U-shaped member 22a formed to have a substantially U-shape, and a second main body portion 22b rotatably supporting the U-shaped member 22a.

The U-shaped member 22a is formed to have a substantially U-shape, and both end portions thereof are coupled to the second main body portion 22b. During the closed state, a central portion of the U-shaped member 22a is disposed on the distal end side of the first stapler gripping member 21. The central portion has a second distal end portion 22c. The second distal end portion 22c is formed to have a substantially parallelepiped shape. The second distal end portion 22c extends in the axial direction C of the opening/closing rotation shaft 23. The staple accommodation portion 4 is provided in the second distal end portion 22c.

The second main body portion 22b is rotatably attached to the first main body portion 21b of the first stapler gripping member 21 by the opening/closing rotation shaft 23. A guide groove 22d having the first main body portion 21b inserted thereinto is formed in the second main body portion 22b. Second engagement groove 22e are respectively formed in a pair of side plate portions 22g facing each other in the axial direction C with the guide groove 22d of the second main body portion 22b therebetween.

The second engagement grooves 22e are grooves penetrating them in the axial direction C. As shown in FIG. 7, the second engagement grooves 22e are formed symmetrically with respect to the central axis O3 of the second stapler gripping member 22. As shown in FIG. 8, the second engagement grooves 22e are inclined toward the downward side B2 as they go from the distal end side to the proximal end side in the axial direction A in a side view during the closed state.

As shown in FIG. 6, the second stapler gripping member 22 has a visual field space (penetration space) 25 penetrating it in the opening/closing direction R between the staple accommodation portion 4 on the distal end side and the opening/closing rotation shaft 23 on the proximal end side. In the present embodiment, the visual field space 25 is a space surrounded by sides of the U-shaped member 22a formed to have a substantially U-shape.

As shown in FIG. 8, the movable pin 27 engages with the first engagement groove 21d and the second engagement grooves 22e and advances and retracts in the axial direction A along the first engagement groove 21d. The distal end of the opening/closing manipulation wire 254 is attached to the movable pin 27. The movable pin 27 advances and retracts in the axial direction A in response to a manipulation of the opening/closing manipulation wire 254, and the second stapler gripping member 22 is opened and closed in association with this as shown in FIGS. 8 and 9.

When the opening/closing manipulation wire 254 is caused to advance to the distal end side, as shown in FIG. 9, the movable pin 27 rotates the second stapler gripping member 22 in an opening direction (R1) about the opening/closing rotation shaft 23 so that the stapler gripping portion 2 is in the open state. When the opening/closing manipulation wire 254 is caused to retract to the proximal end side, as shown in FIG. 8, the movable pin 27 rotates the second stapler gripping member 22 in a closing direction (R2) about the opening/closing rotation shaft 23 so that the stapler gripping portion 2 is in the closed state.

When the stapler gripping portion 2 is in the closed state, as shown in FIG. 8, the staple discharge portion 3 and the staple accommodation portion 4 face each other in the vertical direction B. When the stapler gripping portion 2 is in the closed state, a slight gap P is formed between the staple discharge portion 3 and the staple accommodation portion 4. When the stapler gripping portion 2 is in the closed state, an optical axis A10 of the objective lens 215 passes through the outward side (side on the upward side B1) of the first stapler gripping member 21 and the second stapler gripping member 22. In addition, when the stapler gripping portion 2 is in the closed state, a central axis A20 of the forceps port 214 is at a position not overlapping the first stapler gripping member 21 but overlapping the second stapler gripping member 22 in a front view.

When the stapler gripping portion 2 is in the open state, as shown in FIG. 9, the staple accommodation portion 4 is disposed on the proximal end side from the opening/closing rotation shaft 23. When the stapler gripping portion 2 is in the open state, the staple accommodation portion 4 is disposed on the proximal end side from the staple discharge portion 3. When the stapler gripping portion 2 is in the open state, the optical axis A10 of the objective lens 215 passes through the visual field space 25. In addition, when the stapler gripping portion 2 is in the open state, the central axis A20 of the forceps port 214 passes through the visual field space 25.

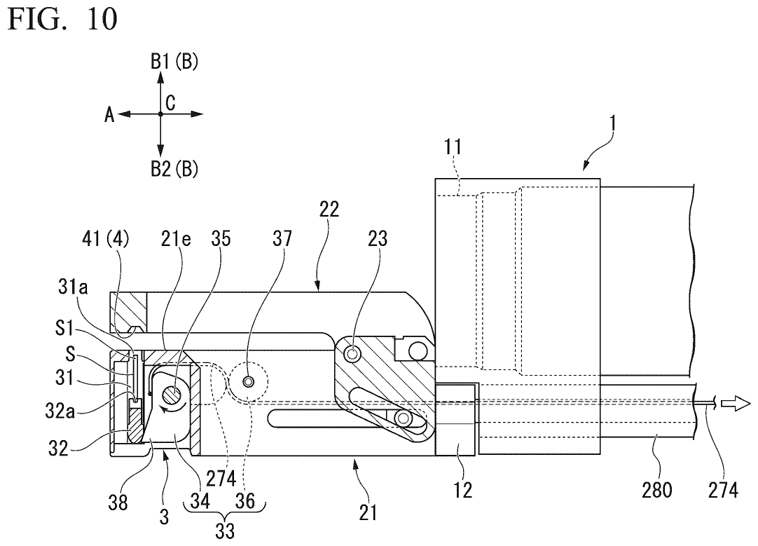

FIG. 10 is a cross-sectional view of the stapler gripping portion 2 including the staple discharge portion 3. FIG. 10 shows a situation before the discharge manipulation wire 274 is dragged.

The staple discharge portion 3 is provided in the first distal end portion 21a of the first stapler gripping member 21 and can store and discharge the staples S. The staple discharge portion 3 has staple storage portions 31, straight advancing members 32, and a rotation member 33.

The staple storage portions 31 are spaces for storing the staples S and provided in the first distal end portion 21a of the first stapler gripping member 21. As shown in FIGS. 6 and 7, two staple storage portions 31 are formed side by side in the axial direction C in the first stapler gripping member 21 and can store two U-shaped staples S.

The staple storage portions 31 open in the vertical direction B in the openings 31a provided on the upper surface 21e of the first distal end portion 21a. The staples S are stored in the staple storage portions 31 through the openings 31a. The staples S are stored in the staple storage portions 31 in a state in which needle tips S1 of the staples S face the upward side B1.

The staple storage portions 31 are each formed to have a rectangular shape in which short sides extend in the axial direction A and long sides extend in the axial direction C in a plan view. In the staples S accommodated in the staple storage portions 31, the needle tips S1 at both ends are arrayed in the axial direction C.

The straight advancing members 32 are members accommodated in bottom portions of the staple storage portions 31 and can move in the vertical direction B in internal spaces of the staple storage portions 31. The straight advancing members 32 have concave portions 32a for supporting the staples S on the upward side B1. The staples S stored in the staple storage portions 31 are fitted into the concave portions 32a.

A first pulley 34 and a second pulley 36 serving as the rotation member 33 are rotatably attached inside the first stapler gripping member 21 and rotate to move the straight advancing members 32 in the vertical direction B. A first rotation shaft 35 of the first pulley 34 and a second rotation shaft 37 of the second pulley 36 extend in the axial direction C and are substantially parallel to the opening/closing rotation shaft 23 of the gripping portion 2.

The first pulley 34 can rotate about the first rotation shaft 35. The distal end of the discharge manipulation wire 274 is coupled to the first pulley 34, and the first pulley 34 rotates when the discharge manipulation wire 274 is dragged. The first pulley 34 has a convex portion (abutment portion) 38 supporting the straight advancing members 32 from the downward side B2 on the distal end side.

The second pulley 36 can rotate about the second rotation shaft 37. The second pulley 36 is a bend pulley disposed on the proximal end side from the first pulley 34 and changes a traveling direction of the discharge manipulation wire 274.

The distal end of the discharge manipulation wire 274 is coupled to the upward side B1 from the first rotation shaft 35 in the first pulley 34. The discharge manipulation wire 274 passes through the second penetration hole 12 via the second pulley 36 from the first pulley 34 and extends to the discharge manipulation portion 270.

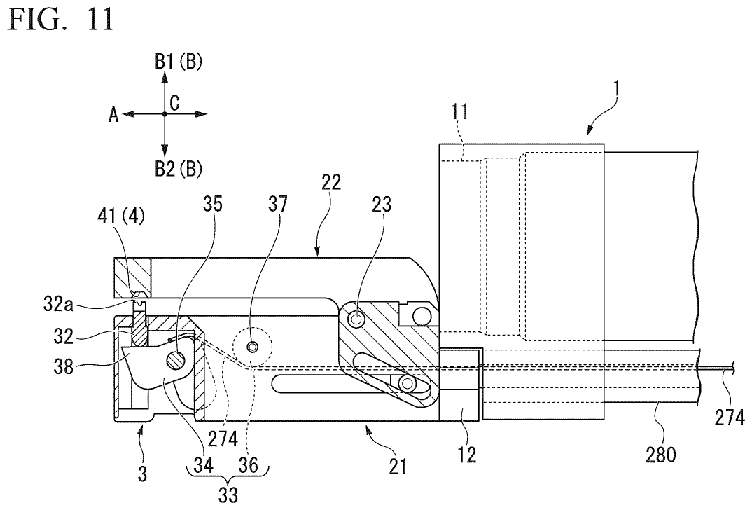

FIG. 11 is a cross-sectional view of the stapler gripping portion 2 in which the discharge manipulation wire 274 is dragged.

When the discharge manipulation wire 274 is dragged, the upward side B1 of the first pulley 34 rotates to the proximal end side, and the downward side B2 of the first pulley 34 rotates to the distal end side. As a result, the convex portion 38 of the first pulley 34 pushes up the straight advancing members 32 to the upward side B1, and the stored staples S are discharged from the openings 31a to the upward side B1.

The staple accommodation portion 4 is provided on a lower surface of the second distal end portion 22c of the second stapler gripping member 22. The staple accommodation portion 4 is provided with a plurality of pockets 41 capable of accommodating the staples S (FIG. 10) discharged from the staple discharge portion 3. In the present embodiment, two U-shaped staples are discharged from the staple discharge portion 3. For this reason, four (FIG. 7) pockets 41 are provided in the staple accommodation portion 4. When the stapler gripping portion 2 is in the closed state, the openings 31a for discharging the staples S (FIG. 10) and the pockets 41 of the staple discharge portion 3 face each other in the vertical direction B.

### [Gripping Forceps (Endoscopic Treatment Tool) 400]

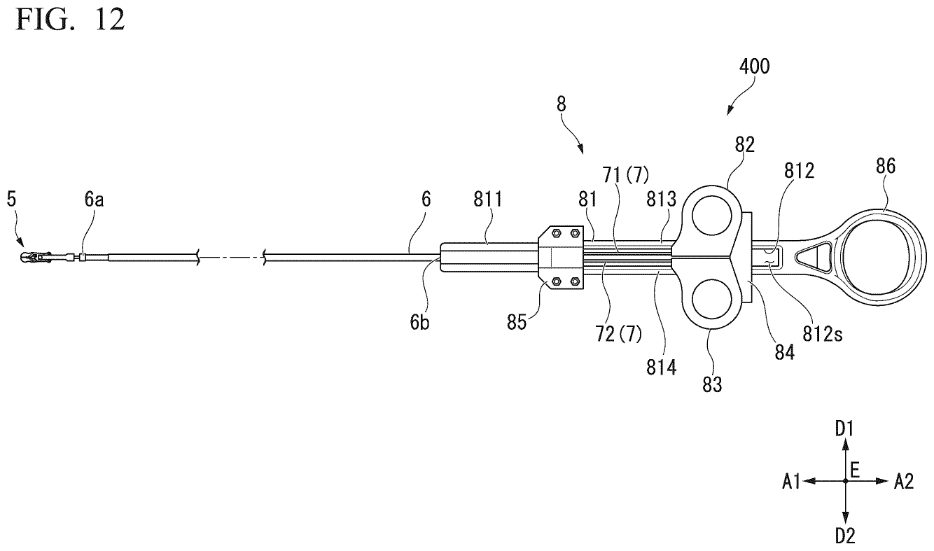

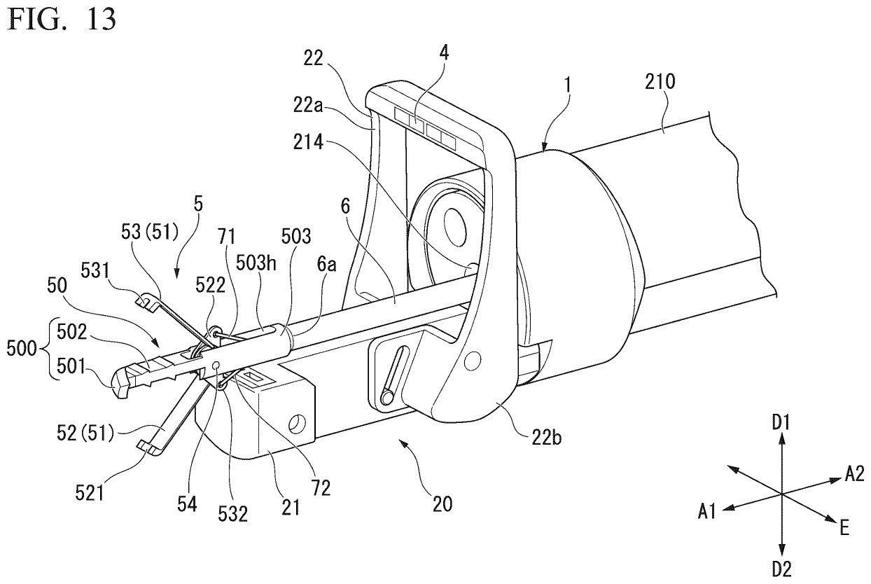

FIG. 12 is an overall view showing the gripping forceps (endoscopic treatment tool) 400 used in the endoscope 200 of the medical system 300. FIG. 13 is a perspective view showing a state in which the medical stapler 100 (refer to FIG. 1) and the gripping forceps 400 are attached to the endoscope 200.

As shown in FIG. 12, the gripping forceps 400 (which will also be referred to as the treatment tool 400) include a forceps gripping portion 5, a forceps sheath (sheath) 6, a forceps manipulation wire 7, and a forceps manipulation portion 8.

In the present embodiment, a longitudinal direction of the gripping forceps 400 is the same direction as the axial direction A. In the following description, the axial direction A will also be referred to as a longitudinal direction A of the gripping forceps 400. In addition, a side to be inserted into the body of a patient in the longitudinal direction A will be referred to as “a distal end side A1”, and the forceps manipulation portion 8 side will be referred to as “a proximal end side A2”. In the gripping forceps 400, the forceps gripping portion 5, the forceps sheath 6, and the forceps manipulation wire 7, and the forceps manipulation portion 8 are disposed from the distal end side A1 to the proximal end side A2 of the gripping forceps 400 in this order. The gripping forceps 400 are inserted into the treatment tool channel 230 from the forceps insertion port 222 provided on the distal end side of the manipulation portion 220 of the endoscope 200 shown in FIG. 1. The gripping forceps 400 inserted from the forceps insertion port 222 are inserted through the treatment tool channel 230 and protrude from the distal ends of the gripping forceps 400 through the forceps port 214 that is the distal end opening of the treatment tool channel 230. The term “patient” used in this specification includes all living things and includes the term “subject”. The patient may be a human or may be an animal.

### [Forceps Gripping Portion 5]

For example, the forceps gripping portion 5 is forceps useful in therapeutic treatment on a patient, such as preventing bleeding of tissue, closing perforations and hemostasis, suturing and contracting internal wounds, traction on a lesion (mucous membrane protuberance), and other surgical treatment. The forceps gripping portion 5 can rotate about a longitudinal axis in the longitudinal direction A. For example, it is easy for the surgeon to be able to rotate the gripping forceps 400 in their entirety by manipulating the forceps manipulation portion 8. The forceps gripping portion 5 includes a rod 50, a forceps piece (jaw) 51, and a coupling pin 54.

Here, the forceps pieces 51 have a first forceps piece (first jaw) 52 and a second forceps piece (second jaw) 53 which are opened and closed with respect to the rod 50. The first forceps piece 52 and the second forceps piece 53 are provided on both sides of the rod 50 with the rod 50 sandwiched therebetween by the coupling pin 54 in a vertical direction D of the gripping forceps 400 perpendicular to the longitudinal direction A and are opened and closed independently from each other. The vertical direction D is an opening/closing direction of the first forceps piece 52 and the second forceps piece 53. For this reason, the vertical direction D will also be referred to as an opening/closing direction D. As shown in FIG. 13, in the vertical direction (opening/closing direction) D, a side where the second forceps piece 53 is provided with respect to the rod 50 will be referred to as an upward side D1, and a side where the first forceps piece 52 is provided with respect to the rod 50 will be referred to as a downward side D2. In addition, a direction perpendicular to the longitudinal direction A and the vertical direction (opening/closing direction) D will be referred to as a width direction E.

### [Rod 50]

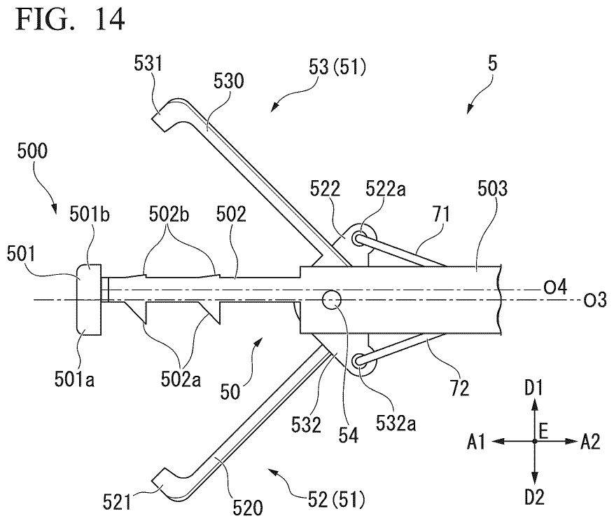

FIG. 14 is a side view showing a state in which the first forceps piece 52 and the second forceps piece 53 of the forceps pieces 51 of the gripping forceps 400 are in the open state with respect to the rod 50.

As shown in FIGS. 13 and 14, the rod 50 is a rod-shaped member extending in the longitudinal direction (axial direction) A and provided between the first forceps piece 52 and the second forceps piece 53 provided on both sides in the vertical direction (opening/closing direction) D. The rod 50 is provided at a distal end 6a of the forceps sheath 6. The rod 50 has a loading portion 500 formed on the distal end side A1, and a coupling portion 503 formed on the proximal end side A2.

For example, the loading portion 500 is a member having a substantially round rod shape formed of a biocompatible material. The loading portion 500 has an outer surface exposed in its entirety and can come into contact with tissue. The loading portion 500 includes a distal end portion 501 and a rod-shaped portion 502.

The distal end portion 501 is provided at the distal end of the rod-shaped portion 502. As shown in FIG. 13, the distal end portion 501 is formed with the vertical direction D as its longitudinal direction so as to have a substantially rhombic shape when viewed from the proximal end side in the longitudinal direction A. The distal end portion 501 is formed non-symmetrically with respect to a central axis O4 of the rod-shaped portion 502 in the longitudinal direction A. Compared to the rod-shaped portion 502, the length of the distal end portion 501 in the vertical direction D is longer than the length of the rod-shaped portion 502. The distal end portion 501 includes a first convex portion 501a and a second convex portion 501b.

As shown in FIG. 14, the first convex portion 501a is a projection part protruding toward the first forceps piece (first jaw) 52 provided on the downward side (first protrusion side) D2 of the loading portion 500 in the vertical direction (protrusion direction) D.

As shown in FIG. 14, the second convex portion 501b is a projection part protruding toward the second forceps piece (second jaw) 53 provided on the upward side (second protrusion side) D1 of the loading portion 500 in the vertical direction (protrusion direction) D.

The distal end portion 501 is manipulated and disposed by the surgeon to be hooked to a living body tissue so that the rod 50 can be locked in the living body tissue. Here, the protrusion length of the first convex portion 501a protruding to the downward side D2 in the vertical direction D is longer than the protrusion length of the second convex portion 501b protruding to the upward side D1 in the vertical direction D.

The rod-shaped portion 502 is a member having a substantially round rod shape, and the distal end portion 501 is provided at the distal end thereof. Here, the rod 50 may be provided in the forceps sheath 6 with the central axis in the longitudinal direction A more eccentric than a central axis O3 of the forceps sheath 6. In the present embodiment, as shown in FIG. 14, the central axis O4 in the longitudinal direction A of the rod-shaped portion 502 provided in the rod 50 is eccentric to the upward side D1 that is a second protrusion side from the central axis O3 of the forceps sheath 6.

As shown in FIG. 14, the rod-shaped portion 502 includes first projection portions 502a on a surface facing the first forceps piece 52 provided on the downward side D2 in the opening/closing direction (vertical direction) D. In addition, the rod-shaped portion 502 includes second projection portions 502b on a surface facing the second forceps piece 53 provided on the upward side D1 in the opening/closing direction D.

As shown in FIG. 14, the first projection portions 502a and the second projection portions 502b are a plurality of projection parts provided in the rod-shaped portion 502. The first projection portions 502a protrude toward the first forceps piece 52 disposed on the downward side D2 in the opening/closing direction D from the rod-shaped portion 502. In addition, the second projection portions 502b protrude toward the second forceps piece 53 disposed on the upward side D1 in the opening/closing direction D from the rod-shaped portion 502.

It is preferable that the first projection portions 502a and the second projection portions 502b be formed to have a size which does not interfere with the first forceps piece 52 and the second forceps piece 53 in the opening/closing direction D. In the present embodiment, the central axis O4 of the rod-shaped portion 502 in the longitudinal direction A is more eccentric to the upward side DI than the central axis O3 of the forceps sheath 6. For this reason, as shown in FIG. 14, the first projection portions 502a are formed to be longer and larger than the second projection portions 502b in the opening/closing direction D. The number of first projection portions 502a and second projection portions 502b are not particularly limited. In addition, the rod-shaped portion 502 does not have to include the first projection portions 502a and the second projection portions 502b.

The coupling portion 503 is coupled to the proximal end of the loading portion 500. Compared to the rod-shaped portion 502 of the loading portion 500, the coupling portion 503 is formed to have a substantially round tubular shape having a diameter dimension larger than the diameter dimension of the rod-shaped portion 502. Both ends of the coupling portion 503 in the longitudinal direction A open such that a first manipulation wire 71 and a second manipulation wire 72 can be inserted therethrough. The proximal end of the coupling portion 503 is coupled to the forceps sheath 6. The distal end of the coupling portion 503 has groove portions 503h on both sides in the vertical direction D.

As shown in FIG. 13, the groove portions 503h are formed to have a U-shape toward the proximal end side A2 in the longitudinal direction A from the distal end of the coupling portion 503.

The rod 50 does not have to include the second convex portion 501b. For example, as shown in FIG. 15, compared to FIG. 14, a rod 50F does not include the second convex portion 501b in a distal end portion 501F of a loading portion 500F.

### [Forceps Piece (Jaw) 51]

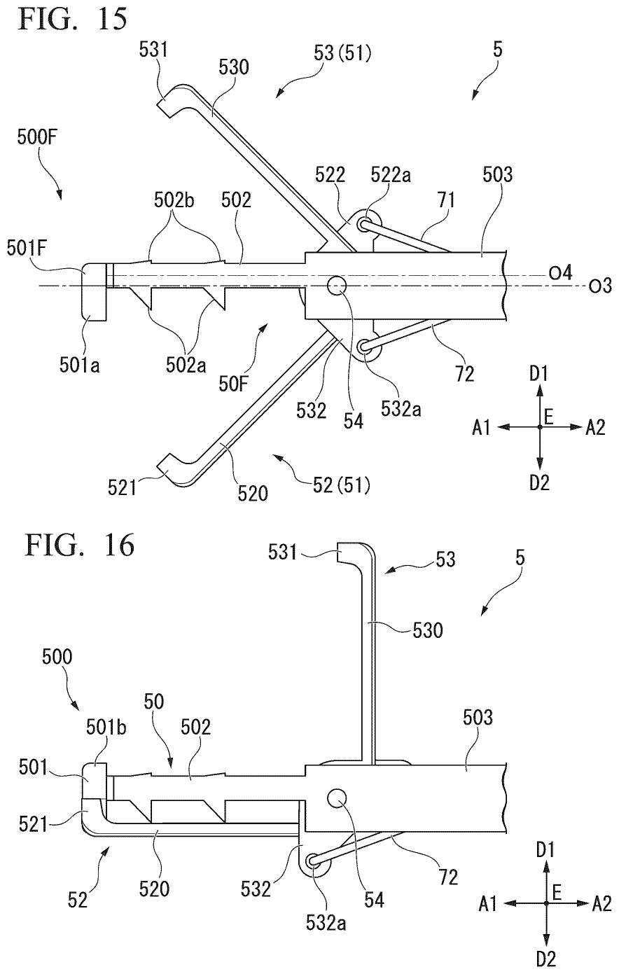

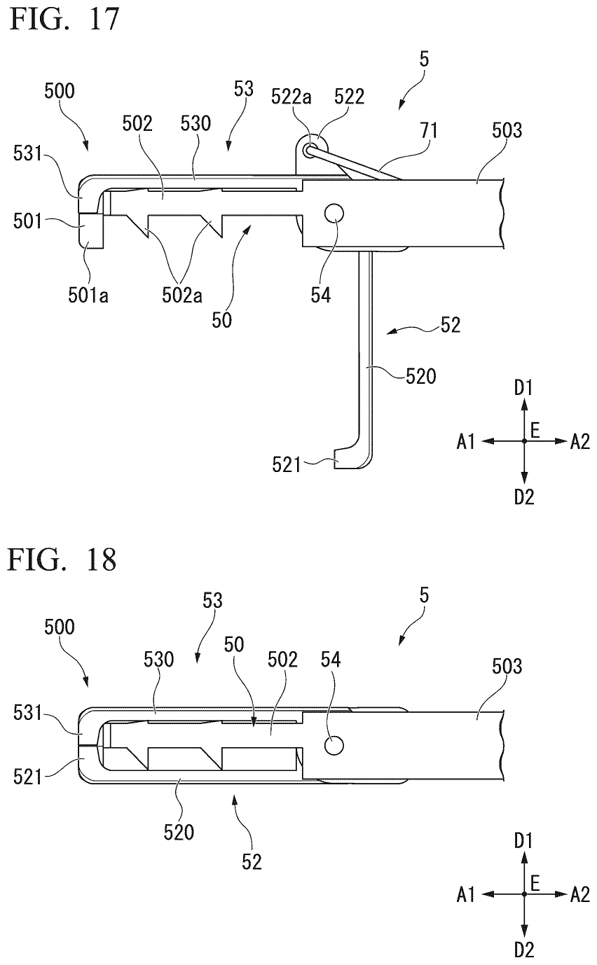

FIG. 16 is a side view showing a state in which the second forceps piece 53 of the forceps pieces 51 of the gripping forceps 400 is in the open state with respect to the rod 50 and the first forceps piece 52 is in the closed state. FIG. 17 is a side view showing a state in which the first forceps piece 52 of the forceps pieces 51 of the gripping forceps 400 is in the open state with respect to the rod 50 and the second forceps piece 53 is in the closed state. FIG. 18 is a side view showing a state in which the first forceps piece 52 and the second forceps piece 53 of the forceps pieces 51 of the gripping forceps 400 are in the closed state with respect to the rod 50.

The forceps piece (jaw) 51 is a member for gripping a living body tissue. For example, the forceps pieces 51 are formed of a metal material such as stainless steel. As shown in FIGS. 14 to 18, the forceps pieces 51 include the first forceps piece (first jaw) 52 and the second forceps piece (second jaw) 53. Each of the first forceps piece 52 and the second forceps piece 53 can perform an opening/closing operation independently with respect to the rod 50. The first forceps piece 52 and the second forceps piece 53 are supported so as to be able to turn in the opening/closing direction (vertical direction) D with the coupling pin 54 as a rotation center. In addition, the first forceps piece 52 and the second forceps piece 53 are disposed symmetrically with respect to the central axis of the forceps pieces 51 in the longitudinal direction A. Here, the central axis of the forceps pieces 51 in the longitudinal direction A substantially coincides with the central axis O3 of the forceps sheath 6 in the longitudinal direction A.

### [First Forceps Piece (First Jaw) 52]

The first forceps piece (first jaw) 52 is provided so as to be openable and closeable on the downward side D2 of the rod 50 in the opening/closing direction D. The first forceps piece 52 extends in the longitudinal direction A in the closed state with respect to the rod 50. The first forceps piece 52 is turnably attached by the coupling pin 54 so as to open away to the downward side D2 with respect to the rod 50. The first forceps piece 52 includes a first arm portion 520 and a first coupling portion 522.

The first arm portion 520 is provided on the distal end side A1 of the first forceps piece 52. For example, the first arm portion 520 is one flat plate made using a material of resin or metal. As shown in FIGS. 16 and 18, the first arm portion 520 is disposed such that the front surface faces the upward side D1 and the rear surface faces the downward side D2 in the closed state with respect to the rod 50. Here, the front surface of the first arm portion 520 is a surface on the inward side coming into contact with a living body tissue and facing the rod 50 in the opening/closing direction D. In addition, the first arm portion 520 is parallel to the rod 50 in the longitudinal direction A in a state in which the first forceps piece 52 is closed with respect to the rod 50. The first arm portion 520 may be formed to have a substantially cup shape in which the rear surface becomes a bottom surface in the closed state with respect to the rod 50. The first arm portion 520 includes an engagement portion 521 on the distal end side A1.

The engagement portion 521 is provided on the distal end side A1 of the first arm portion 520. As shown in FIGS. 16 and 18, the engagement portion 521 is formed by bending the distal end of the first arm portion 520 such that the distal end of the engagement portion 521 faces the upward side D1 in a state in which the first forceps piece 52 is closed with respect to the rod 50. With this constitution, the number of components required for the engagement portion 521 can be reduced, and the manufacturing steps can be facilitated.

The engagement portion 521 is formed to have a claw shape having a distal end divided into two parts and engages with the first convex portion 501a of the distal end portion 501 provided at the distal end of the rod-shaped portion 502 of the rod 50. With this constitution, the first forceps piece 52 can reliably pinch a living body tissue when being closed with respect to the rod 50.

The first coupling portion 522 is provided on the proximal end side A2 of the first forceps piece 52. The first coupling portion 522 is formed to have a substantially plate shape having a surface in the width direction E. The first coupling portion 522 is coupled to the proximal end portion of the first arm portion 520. The first coupling portion 522 includes a first penetration hole 522a penetrating it in the width direction E. As shown in FIGS. 14 and 17, in a state in which the first forceps piece 52 is opened with respect to the rod 50, the first coupling portion 522 is thrust into the groove portions 503h provided at the distal end of the coupling portion 503 of the rod 50 so as to be disposed on the upward side D1 from the coupling portion 503 without overlapping the coupling portion 503 of the rod 50 when the first penetration hole 522a is viewed in the width direction E.

For example, the first forceps piece 52 may have a marker which is visually distinguishable from the second forceps piece 53. The marker is not particularly limited and need only be distinguishable from the second forceps piece 53 by color, pattern, shape, and the like.

### [Second Forceps Piece (Second Jaw) 53]

The second forceps piece (second jaw) 53 is provided so as to be openable and closeable on the upward side D1 on a side opposite to the first forceps piece 52 with the rod 50 sandwiched therebetween in the opening/closing direction D. The second forceps piece 53 extends in the longitudinal direction A in the closed state with respect to the rod 50. The second forceps piece 53 is turnably attached by the coupling pin 54 so as to open away to the upward side D1 with respect to the rod 50. The second forceps piece 53 includes a second arm portion 530 and a second coupling portion 532.

The second arm portion 530 is provided on the distal end side A1 of the second forceps piece 53. For example, the second arm portion 530 is one flat plate made using a material of resin or metal. As shown in FIGS. 17 and 18, the second arm portion 530 is disposed such that the front surface faces the downward side D2 and the rear surface faces the upward side D1 in the closed state with respect to the rod 50. Here, the front surface of the second arm portion 530 is a surface on the inward side coming into contact with a living body tissue and facing the rod 50 in the opening/closing direction D. In addition, the second arm portion 530 is parallel to the rod 50 in the longitudinal direction A in a state in which the second forceps piece 53 is closed with respect to the rod 50. The second arm portion 530 may be formed to have a substantially cup shape in which the rear surface becomes a bottom surface in the closed state with respect to the rod 50. The second arm portion 530 includes an engagement portion 531 on the distal end side A1.

The engagement portion 531 is provided on the distal end side A1 of the second arm portion 530. As shown in FIGS. 17 and 18, the engagement portion 531 is formed by bending the distal end of the second arm portion 530 such that the distal end of the engagement portion 531 faces the downward side D2 in a state in which the second forceps piece 53 is closed with respect to the rod 50. With this constitution, the number of components required for the engagement portion 531 can be reduced, and the manufacturing steps can be facilitated.

The engagement portion 531 is formed to have a claw shape having a distal end divided into two parts and engages with the second convex portion 501b of the distal end portion 501 provided at the distal end of the rod-shaped portion 502 of the rod 50. With this constitution, the second forceps piece 53 can reliably pinch a living body tissue when being closed with respect to the rod 50.

The second coupling portion 532 is provided on the proximal end side A2 of the second forceps piece 53. The second coupling portion 532 is formed to have a substantially plate shape having a surface in the width direction E. The second coupling portion 532 is coupled to the proximal end portion of the second arm portion 530. The second coupling portion 532 includes a second penetration hole 532a penetrating it in the width direction E. As shown in FIGS. 14 and 16, in a state in which the second forceps piece 53 is opened with respect to the rod 50, the second coupling portion 532 is thrust into the groove portions 503h provided at the distal end of the coupling portion 503 of the rod 50 so as to be disposed on the downward side D2 from the coupling portion 503 without overlapping the coupling portion 503 of the rod 50 when the second penetration hole 532a is viewed in the width direction E.

The opening/closing angle between the first forceps piece 52 and the second forceps piece 53 with respect to the rod 50 is not particularly limited. However, it is preferably set to 90 degrees or greater such that a living body tissue is likely to be gripped even when the longitudinal direction A of the gripping forceps 400 is a direction nearly parallel to the surface of the stomach wall.

### [Coupling Pin 54]

As shown in FIGS. 14 to 18, the coupling pin 54 is attached to the distal end side A1 of the coupling portion 503 of the rod 50. The coupling pin 54 couples the first coupling portion 522 of the first forceps piece 52 and the second coupling portion 532 of the second forceps piece 53 with respect to the coupling portion 503 of the rod 50 in the width direction E. Specifically, the coupling pin 54 is attached to the distal end side A1 from the first penetration hole 522a in the first coupling portion 522 in a state in which the first forceps piece 52 is closed with respect to the rod 50. In addition, the coupling pin 54 is attached to the distal end side A1 from the second penetration hole 532a in the second coupling portion 532 in a state in which the second forceps piece 53 is closed with respect to the rod 50. The first forceps piece 52 and the second forceps piece 53 are opened and closed independently in the opening/closing direction D with the coupling pin 54 as a rotation center.

### [Forceps Sheath 6]

The forceps sheath 6 is a long member having the central axis O3 (refer to FIG. 14) and extending from the distal end 6a to a proximal end 6b. The forceps sheath 6 is flexible so that the shape can easily change along the bent shape of a lumen tissue or the like inside a lumen. The forceps sheath 6 has an outer diameter which can be inserted into the treatment tool channel 230 of the endoscope 200. The distal end 6a of the forceps sheath 6 can project and retract through the forceps port 214 that is the distal end opening of the treatment tool channel 230 in a state of being inserted into the treatment tool channel 230. The distal end 6a of the forceps sheath 6 is connected to the rod 50. The forceps sheath 6 is formed of a material having insulating properties, for example, a fluorine resin such as polytetrafluoroethylene (PTFE) or a resin material such as high-density polyethylene (HDPE). In addition, the first manipulation wire 71 and the second manipulation wire 72 of the forceps manipulation wire 7 are inserted through the forceps sheath 6.

### [Forceps Manipulation Wire 7]

As shown in FIGS. 12 to 18, the forceps manipulation wire 7 includes the first manipulation wire 71 and the second manipulation wire 72. The first manipulation wire 71 and the second manipulation wire 72 are inserted through an internal space (not shown) of the forceps sheath 6.

The first manipulation wire 71 is inserted through the inside of the forceps sheath 6 so as to be able to advance and retract in the longitudinal direction A. As shown in FIGS. 14 and 17, the distal end portion of the first manipulation wire 71 is coupled to the first penetration hole 522a of the first coupling portion 522 provided in the first forceps piece 52. If the first manipulation wire 71 advances to the distal end side A1 in the longitudinal direction A, the distal end portion of the first manipulation wire 71 sticks out to the upward side D1 from the groove portions 503h provided in the coupling portion 503 of the rod 50 provided at the distal end 6a of the forceps sheath 6 and rotates the first coupling portion 522. Consequently, the first arm portion 520 of the first forceps piece 52 turns to the downward side D2 about the coupling pin 54. The proximal end portion of the first manipulation wire 71 is fixed to a first slider 82 of the forceps manipulation portion 8. For example, the first manipulation wire 71 may be formed of a metal solid wire or a metal stranded wire. In addition, the outer circumferential surface of the first manipulation wire 71 may be covered with a non-conductive member or the like. The first manipulation wire 71 is fixed to the first penetration hole 522a of the first coupling portion 522 by various known methods, for example, a method such as bonding or welding.

The second manipulation wire 72 is inserted through the inside of the forceps sheath 6 so as to be able to advance and retract in the longitudinal direction A. As shown in FIGS. 14 and 16, the distal end portion of the second manipulation wire 72 is coupled to the second penetration hole 532a of the second coupling portion 532 provided in the second forceps piece 53. If the second manipulation wire 72 advances to the distal end side A1 in the longitudinal direction A, the distal end portion of the second manipulation wire 72 sticks out to the downward side D2 from the groove portions 503h provided in the coupling portion 503 of the rod 50 provided at the distal end 6a of the forceps sheath 6 and rotates the second coupling portion 532. Consequently, the second arm portion 530 of the second forceps piece 53 turns to the upward side D1 about the coupling pin 54. The proximal end portion of the second manipulation wire 72 is fixed to a second slider 83 of the forceps manipulation portion 8. For example, the second manipulation wire 72 may be formed of a metal solid wire or a metal stranded wire. In addition, the outer circumferential surface of the second manipulation wire 72 may be covered with a non-conductive member or the like. The second manipulation wire 72 is fixed to the second penetration hole 532a of the second coupling portion 532 by various known methods, for example, a method such as bonding or welding.

With the foregoing constitution, when the first manipulation wire 71 and the second manipulation wire 72 advance and retract in the longitudinal direction A, the first forceps piece 52 and the second forceps piece 53 is subjected to an opening/closing manipulation.

### [Forceps Manipulation Portion (Handle) 8]

As shown in FIG. 12, the forceps manipulation portion 8 is provided on the proximal end side A2 of the forceps sheath 6. The forceps manipulation portion 8 manipulates the forceps gripping portion 5 with the first manipulation wire 71 and the second manipulation wire 72 sandwiched therebetween. The forceps manipulation portion 8 includes a forceps manipulation portion main body 81, the first slider 82, the second slider 83, a simultaneous opening/closing assistance portion (abutment portion) 84, a forceps rotation portion 85, and a finger hooking portion 86.

The forceps manipulation portion main body 81 is formed to have a rod shape extending in the longitudinal direction A. The forceps manipulation portion main body 81 includes a sheath fixing portion 811 and a penetration hole 812.

The sheath fixing portion 811 is provided on the distal end side A1 of the forceps manipulation portion main body 81. In the sheath fixing portion 811, the proximal end 6b of the forceps sheath 6 can be attached inside the sheath fixing portion 811 by a known method in the related art. The sheath fixing portion 811 connects the forceps sheath 6 and the forceps manipulation portion main body 81.

As shown in FIG. 12, the penetration hole 812 is a long hole extending along the shape of the forceps manipulation portion main body 81. The penetration hole 812 penetrates the forceps manipulation portion main body 81 in the width direction E. In the forceps manipulation portion main body 81, by providing the penetration hole 812, a first side portion 813 in which the first slider 82 can slide in the longitudinal direction A on the upward side D1 in the vertical direction D and a second side portion 814 in which the second slider 83 can slide in the longitudinal direction A on the downward side D2 in the vertical direction D are formed. In a penetration space 812s of the penetration hole 812, the first manipulation wire 71 and the second manipulation wire 72 inserted through the forceps sheath 6 extend in the longitudinal direction A.

The first slider 82 is attached so as to be able to slide in the longitudinal direction A in the first side portion 813 of the forceps manipulation portion main body 81. The first slider 82 may be attached by being inserted through the first side portion 813 or may be attached by assembling a plurality of components in the first side portion 813 of the forceps manipulation portion main body 81. The first slider 82 is formed to have a substantially ring shape on the upward side D1 from the center of the forceps manipulation portion main body 81 so that it is easy for the surgeon to hook his/her finger. In addition, the proximal end portion of the first manipulation wire 71 is fixed to the first slider 82. The surgeon causes the first manipulation wire 71 to advance and retract by causing the first slider 82 to advance and retract relatively with respect to the forceps manipulation portion main body 81. Specifically, if the first slider 82 is caused to advance to the distal end side A1 in the longitudinal direction A along the forceps manipulation portion main body 81, the first manipulation wire 71 advances to the distal end side A1 in the longitudinal direction A in association therewith. Consequently, the first forceps piece 52 fixed to the distal end portion of the first manipulation wire 71 turns to the downward side D2 about the coupling pin 54. With this constitution, if the first slider 82 is caused to slide in the longitudinal direction A in the forceps manipulation portion main body 81, the first forceps piece 52 can be driven and subjected to an opening/closing manipulation in the opening/closing direction D.

The second slider 83 is attached so as to be able to slide in the longitudinal direction A in the second side portion 814 of the forceps manipulation portion main body 81. The second slider 83 may be attached by being inserted through the second side portion 814 or may be attached by assembling a plurality of components in the second side portion 814 of the forceps manipulation portion main body 81. The second slider 83 is formed to have a substantially ring shape on the downward side D2 from the center of the forceps manipulation portion main body 81 so that it is easy for the surgeon to hook his/her finger. In addition, the proximal end portion of the second manipulation wire 72 is fixed to the second slider 83. The surgeon causes the second manipulation wire 72 to advance and retract by causing the second slider 83 to advance and retract relatively with respect to the forceps manipulation portion main body 81. Specifically, if the second slider 83 is caused to advance to the distal end side A1 in the longitudinal direction A along the forceps manipulation portion main body 81, the second manipulation wire 72 advances to the distal end side A1 in the longitudinal direction A in association therewith. Consequently, the second forceps piece 53 fixed to the distal end portion of the second manipulation wire 72 turns to the upward side D1 about the coupling pin 54. With this constitution, if the second slider 83 is caused to slide in the longitudinal direction A in the forceps manipulation portion main body 81, the second forceps piece 53 can be driven and subjected to an opening/closing manipulation in the opening/closing direction D. Each of the first slider 82 and the second slider 83 independently slides in the longitudinal direction A in the forceps manipulation portion main body 81.

The simultaneous opening/closing assistance portion (abutment portion) 84 is attached in a manner of straddling the first side portion 813 and the second side portion 814 in the forceps manipulation portion main body 81. The simultaneous opening/closing assistance portion 84 can abut the proximal end portion of the first slider 82 and the proximal end portion of the second slider 83. If the simultaneous opening/closing assistance portion 84 is moved to the distal end side A1 in the longitudinal direction A in the forceps manipulation portion main body 81, it simultaneously abuts the proximal end portion of the first slider 82 and the proximal end portion of the second slider 83 and causes the first slider 82 and the second slider 83 to simultaneously slide to the distal end side A1 in the longitudinal direction A. Consequently, the simultaneous opening/closing assistance portion 84 can simultaneously open the first forceps piece 52 and the second forceps piece 53 with the first slider 82 and the second slider 83 sandwiched therebetween.

The forceps rotation portion 85 is attached to the forceps manipulation portion main body 81. It is easy for the surgeon to be able to rotate the gripping forceps 400 by rotating the forceps rotation portion 85 in the axial direction of the gripping forceps 400 using one hand which does not manipulate the slider while manipulating the first slider 82 and the second slider 83 using the other hand. A place to which the forceps rotation portion 85 is attached is not particularly limited. However, it is preferable that the forceps rotation portion 85 be attached to a position not interfering with the first slider 82 and the second slider 83 which slide in the forceps manipulation portion main body 81.

The finger hooking portion 86 is a finger hooking part having a substantially ring shape formed on the proximal end side A2 of the forceps manipulation portion main body 81. The surgeon can cause the gripping forceps 400 to retract to the proximal end side A2 by holding the finger hooking portion 86 and hauling the gripping forceps 400 to the proximal end side A2.

### [Method for Using Medical Stapler 100 Using Gripping Forceps 400]

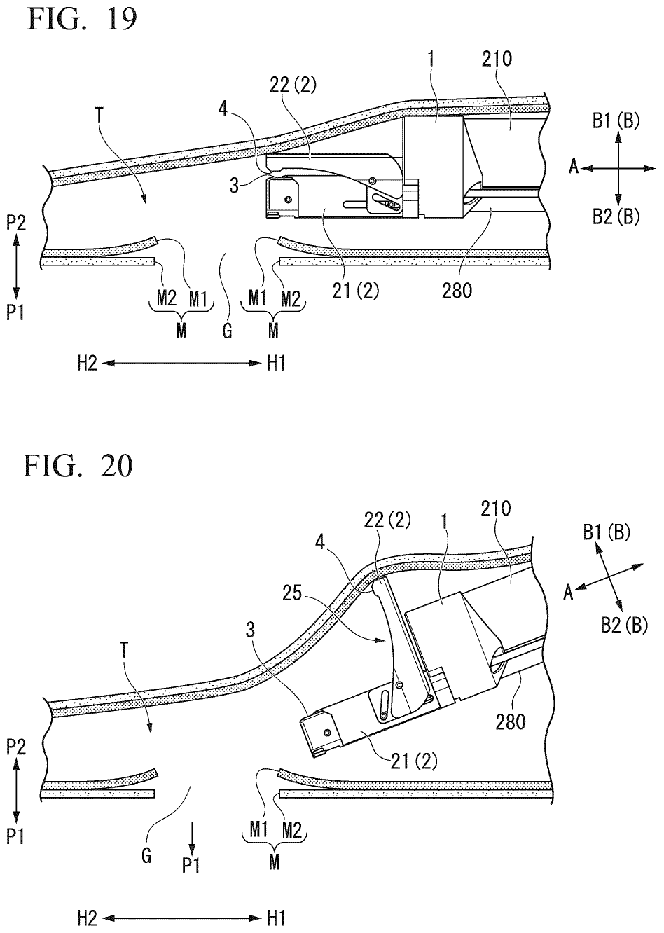

Next, a method for using the medical stapler 100 using the gripping forceps 400 will be described with reference to FIGS. 19 to 31. Specifically, a method for performing suturing treatment for a defective part (resection hole) G, which has been formed by endoscopic therapy after full-thickness resection of a lesion formed in the stomach, will be described.

### <Marking Step>

FIG. 19 is a view showing a state in which the endoscope 200 is brought close to a lesion.

A surgeon or an assistant (who will hereinafter be simply referred to as “a surgeon”) inserts the insertion portion 210 of the endoscope 200 having the medical stapler 100 mounted therein from a port that is a natural opening and brings the distal end portion 211 closer to a treatment target T on the downward side B2 from the endoscope 200. The surgeon inserts a marking treatment tool such as a high-frequency knife into the treatment tool channel 230 by a known method in the related art and performs marking around a lesion of a treatment target T. The marking treatment tool may be high-frequency forceps, a high-frequency snare, a heating element such as a heat probe, an ultrasonic device, or the like. The surgeon forms a pair of markings in peripheral tissues on both sides of a lesion by pressing a knife of the high-frequency knife against the peripheral tissues surrounding the lesion in the treatment target T and cauterizing it.

### <Full-Thickness Resecting Step>

The surgeon performs full-thickness resection of a stomach wall (tissue) M including a mucous membrane layer M1 and a muscle layer M2 formed on an abdominal cavity side (muscle layer side) P1 from the mucous membrane layer M1 along the markings using the marking treatment tool. Consequently, air inside the stomach escapes to the abdominal cavity side P1 through the defective part (resection hole) G formed in the stomach wall M by full-thickness resection, and therefore the stomach collapses and becomes deformed (contracts) as shown in FIG. 19.

### <Disposing Step>

FIG. 20 is a view showing a disposing step.

The surgeon disposes the medical stapler 100 provided on the distal end side of the endoscope 200 in an approaching direction toward the defective part G of the treatment target T. Here, the approaching direction is the same direction as the axial direction (longitudinal direction) A of the medical stapler 100. Since the inside of the stomach contracts in the full-thickness resecting step, the approaching direction becomes a direction in a tangent direction H nearly parallel to the surface of the stomach wall M. The abdominal cavity side (muscle layer side) P1 is the same direction as the downward side B2 in the vertical direction B in a state in which the medical stapler 100 is directed in the approaching direction. The defective part G has a first end portion Ta that is an end portion on a near side H1 in the tangent direction H at a circumferential edge. In addition, the defective part G has a second end portion Tb that is an end portion on a far side H2 in the tangent direction H at the circumferential edge.

Next, in order to observe the treatment target T through the objective lens 215 in a state in which the medical stapler 100 is mounted in the distal end portion 211 of the endoscope 200, the surgeon tilts the insertion portion 210 such that the distal end portion 211 of the insertion portion 210 faces the defective part G. Since the optical axis A10 of the objective lens 215 passes through the visual field space 25 when the stapler gripping portion 2 is in the open state, the surgeon can observe the treatment target T with the image capturing unit of the endoscope 200 sandwiched therebetween. However, in a state in which the inside of the stomach has contracted, it is difficult for the surgeon to align the approaching direction with a direction in which the treatment target T perpendicular to the tangent direction H is likely to be observed. For this reason, the approaching direction is still a direction in the tangent direction H. At the circumferential edge of the defective part G, the muscle layer M2 exposed to the first end portion Ta on the near side H1 provided on the near side H1 in the tangent direction His hidden by the mucous membrane layer M1. For this reason, it is difficult for the surgeon to accurately visually recognize the first end portion Ta. In this state, the surgeon causes the opening/closing manipulation wire 254 to advance by manipulating the opening/closing manipulation portion 250 of the endoscope 200 to cause the stapler gripping portion 2 to be in the open state.

### <Inserting Step>

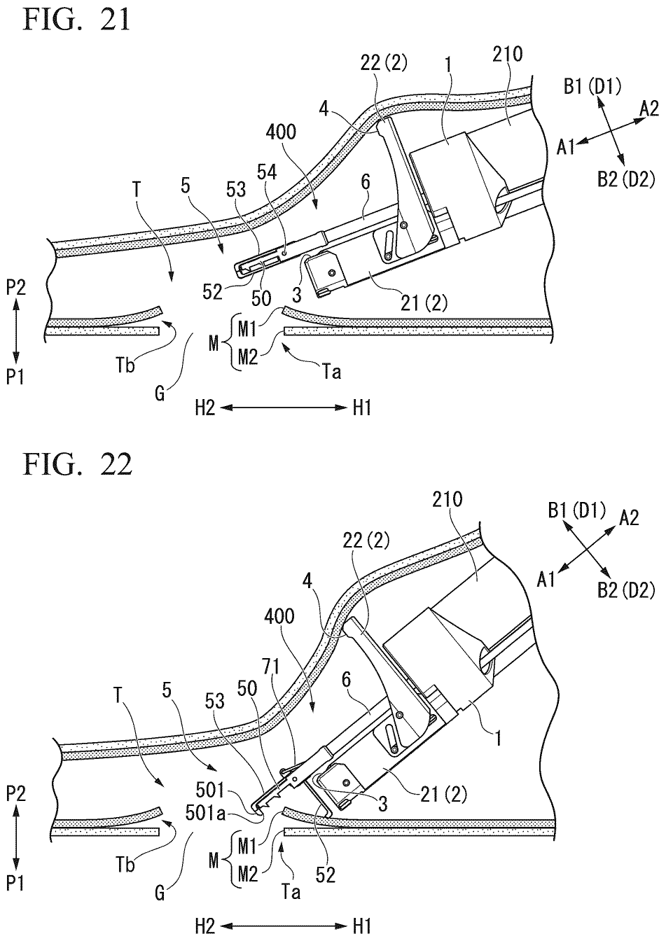

FIG. 21 is a view showing an inserting step of the gripping forceps 400.

The surgeon inserts the gripping forceps 400 as a drawing treatment tool into the treatment tool channel 230. Specifically, the surgeon inserts the gripping forceps 400 toward the treatment tool channel 230 from the forceps insertion port 222 provided on the distal end side of the manipulation portion 220 of the endoscope 200. The gripping forceps 400 inserted from the forceps insertion port 222 are inserted through the treatment tool channel 230 and protrude from the distal ends of the gripping forceps 400 to the forceps port 214 that is the distal end opening of the treatment tool channel 230. As shown in FIG. 21, the gripping forceps 400 are disposed near the center of the defective part G in the approaching direction.

### <Gripping Forceps Disposing Step>

In the disposing step, the surgeon disposes the forceps gripping portion 5 by rotating it about the longitudinal axis in the longitudinal direction A. For example, it is easy for the surgeon to be able to rotate the forceps gripping portion 5 of the gripping forceps 400 by manipulating the forceps rotation portion 85 provided in the forceps manipulation portion 8. In the present embodiment, in the forceps gripping portion 5, the first forceps piece 52 provided on the downward side D2 with respect to the rod 50 is disposed on the downward side B2 of the cap 1 in the vertical direction B as shown in FIGS. 21 to 29. At this time, the opening/closing direction D becomes the same direction as the vertical direction B. The first forceps piece 52 is disposed on the abdominal cavity side (muscle layer side) P1 in the same direction as the downward side B2 in the vertical direction B. When the opening/closing direction D is the same direction as the vertical direction B, a width direction E becomes the same direction as the axial direction C. When the first forceps piece 52 has a marker which is visually distinguishable from the second forceps piece 53, it is easy for the surgeon to dispose the first forceps piece 52 on the abdominal cavity side P1 with the image capturing unit of the endoscope 200 sandwiched therebetween.

### <First Opening/Closing Step>

FIG. 22 is a view showing a first opening/closing step of the gripping forceps 400.

As shown in FIG. 22, the surgeon opens and closes the first forceps piece 52 near the first end portion Ta of the defective part G that is the treatment target T. More specifically, the surgeon causes the first slider 82 of the forceps manipulation portion 8 to slide to the distal end side A1 in the longitudinal direction A in the forceps manipulation portion main body 81. Consequently, the first manipulation wire 71 fixed to the first slider 82 moves to the distal end side A1 in the longitudinal direction A inside the forceps sheath 6. The distal end portion of the first manipulation wire 71 is coupled to the first penetration hole 522a of the first coupling portion 522. The distal end portion of the first manipulation wire 71 sticks out to the upward side D1 from the groove portions 503h provided in the coupling portion 503 of the rod 50 and rotates the first coupling portion 522. Consequently, the first forceps piece 52 turns to the downward side D2 with the coupling pin 54 as a rotation center. The first forceps piece 52 turns to the downward side D2 in the opening/closing direction D away from the distal end side A1 of the rod 50. As a result, the first forceps piece 52 is in the open state with respect to the rod 50.

### <Thrusting Step>

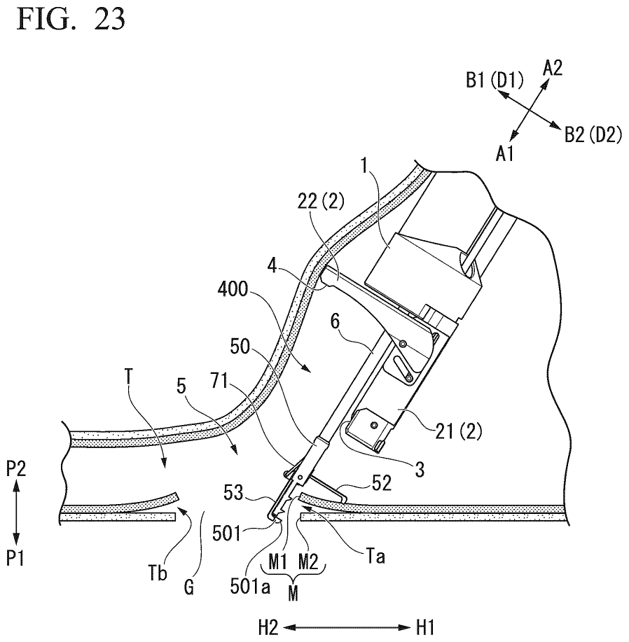

FIG. 23 is a view showing a thrusting step of the gripping forceps 400.

As shown in FIG. 23, in a state in which the first forceps piece 52 is in the open state with respect to the rod 50, the surgeon thrusts the rod 50 provided at the distal end 6a of the forceps sheath 6 and disposed in the longitudinal direction (approaching direction) A into the defective part (resection hole) G. In this state, the surgeon moves the first forceps piece 52 and the rod 50 in the open state toward the first end portion Ta of the defective part G to be gripped and positions the first end portion Ta between the first forceps piece 52 and the rod 50. At this time, the surgeon disposes the distal end portion 501 of the rod 50 on the abdominal cavity side P1 from the muscle layer M2 exposed to the first end portion Ta.

### <Hooking Step>

The distal end portion 501 of the rod 50 includes the first convex portion 501a on the downward side D2 in the vertical direction D. The surgeon hooks the first convex portion 501a of the distal end portion 501 onto the muscle layer M2 formed on the abdominal cavity side P1 from the mucous membrane layer M1 such that the rod 50 is locked in the stomach wall M of the stomach. The surgeon may rotate the forceps gripping portion 5 in the midst of a hooking step.

### <Pulling Step>

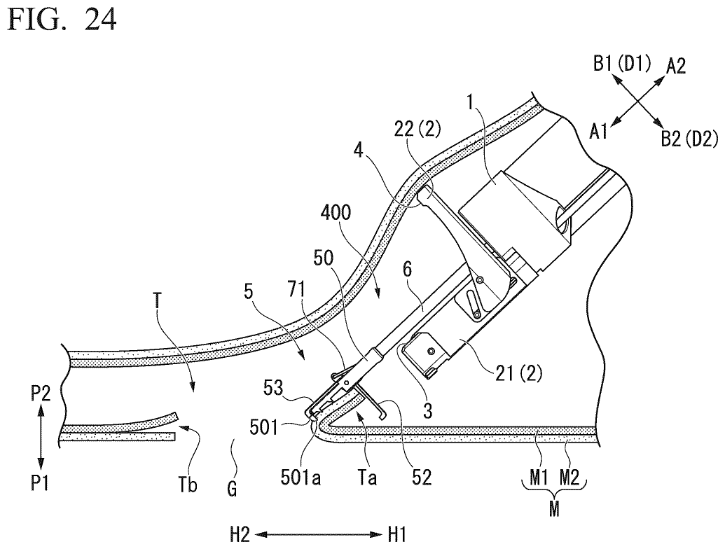

FIG. 24 is a view showing a pulling step of the gripping forceps 400.

As shown in FIG. 24, in a state in which the first convex portion 501a of the distal end portion 501 of the rod 50 is hooked and locked in the muscle layer M2 of the first end portion Ta, the surgeon pulls it from the abdominal cavity side P1 to a stomach inward side (mucous membrane layer side) P2 opposite to the abdominal cavity side P1.

Here, the protrusion length of the first convex portion 501a protruding to the downward side D2 in the vertical direction D is longer than the protrusion length of the second convex portion 501b protruding to the upward side D1 in the vertical direction D. For this reason, it is easy for the surgeon to pull the first end portion Ta entirely with the muscle layer M2 even if the first end portion Ta of the defective part G on the near side H1 cannot be accurately visually recognized.

### <First Gripping Step>

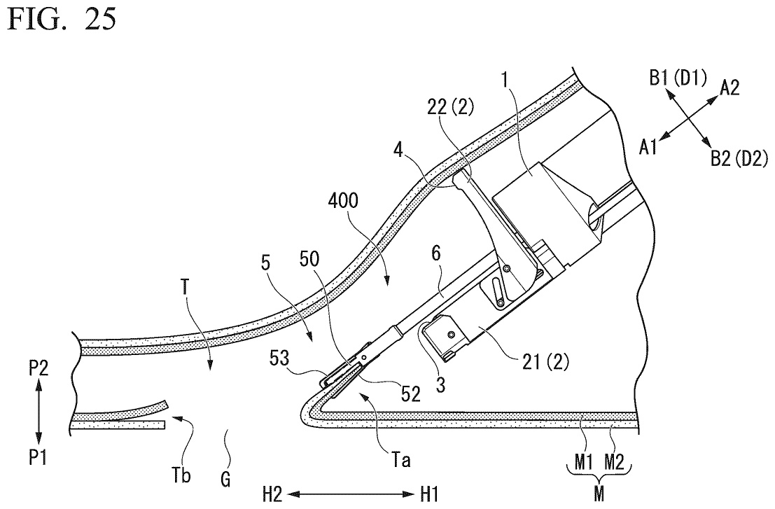

FIG. 25 is a view showing a first gripping step of the gripping forceps 400.

If the surgeon can confirm that the first end portion Ta pulled by the first convex portion 501a of the distal end portion 501 of the rod 50 is positioned between the first forceps piece 52 and the rod 50, the first slider 82 of the forceps manipulation portion 8 is caused to slide to the proximal end side A2 in the longitudinal direction A in the forceps manipulation portion main body 81. Consequently, the first manipulation wire 71 fixed to the first slider 82 moves to the proximal end side A2 in the longitudinal direction A inside the forceps sheath 6. The distal end portion of the first manipulation wire 71 rotates the first coupling portion 522 so as to be drawn to the groove portions 503h provided in the coupling portion 503. Consequently, the first forceps piece 52 turns to the upward side D1 with the coupling pin 54 as a rotation center. The first forceps piece 52 turns to the upward side D1 in the opening/closing direction D so as to approach the distal end side A1 of the rod 50. As a result, the first forceps piece 52 is in the closed state with respect to the rod 50, and the rod 50 and the first forceps piece 52 grip the first end portion Ta of the defective part G. At this time, in the first end portion Ta of the defective part G, both the muscle layer M2 and the mucous membrane layer M1 are gripped by the first forceps piece 52 and the rod 50. The surgeon may rotate the forceps gripping portion 5 in the midst of the first gripping step. It is easy for the surgeon to be able to rotate the forceps rotation portion 85 in the axial direction of the gripping forceps 400 using one hand which does not manipulate the slider while manipulating the first slider 82 and the second slider 83 using the other hand. For this reason, even if the stomach wall M of the stomach is in a direction in which it is difficult for the gripping forceps 400 to grip it, the surgeon can perform adjustment by rotating the forceps gripping portion 5 to make it easier to grasp it.

### <Regrasping Step>

In a regrasping step after the first gripping step has been executed, the surgeon can regrip the first end portion Ta of the defective part G by manipulating the forceps manipulation portion 8 again to turn the first forceps piece 52 and shift the first forceps piece 52 to the open state with respect to the rod 50.

### <Dragging Step>

While the state in which the first end portion Ta of the defective part G is gripped by the first forceps piece 52 and the rod 50 is maintained, the surgeon causes the gripping forceps 400 and the endoscope 200 in their entirety to advance to a location near the second end portion Tb on the far side H2 that is the end portion on the far side H2 in the tangent direction H at the circumferential edge of the defective part G. In this process, the surgeon maintains the closed state of the first forceps piece 52 and the rod 50.

### <Second Opening/Closing Step>

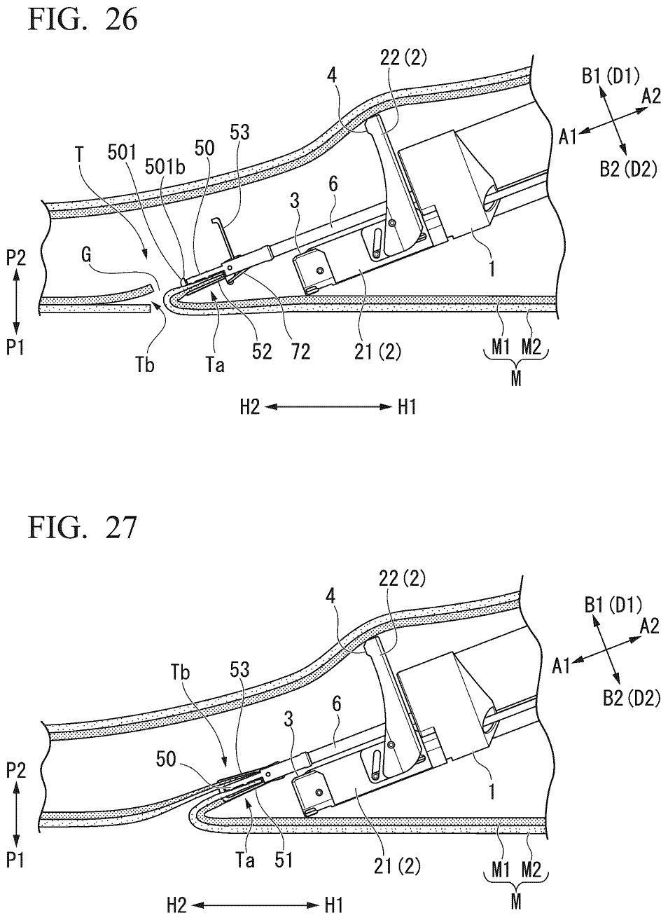

FIG. 26 is a view showing a second opening/closing step of the gripping forceps 400.

As shown in FIG. 26, the surgeon opens and closes the second forceps piece 53 near the second end portion Tb. More specifically, the surgeon causes the second slider 83 of the forceps manipulation portion 8 to slide to the distal end side A1 in the longitudinal direction A in the forceps manipulation portion main body 81. Consequently, the second manipulation wire 72 fixed to the second slider 83 moves to the distal end side A1 in the longitudinal direction A inside the forceps sheath 6. The distal end portion of the second manipulation wire 72 is coupled to the second penetration hole 532a of the second coupling portion 532. The distal end portion of the second manipulation wire 72 sticks out to the downward side D2 from the groove portions 503h provided in the coupling portion 503 of the rod 50 and rotates the second coupling portion 532. Consequently, the second forceps piece 53 turns to the upward side D1 with the coupling pin 54 as a rotation center. The second forceps piece 53 turns to the upward side D1 in the opening/closing direction D away from the distal end side A1 of the rod 50. As a result, the second forceps piece 53 is in the open state with respect to the rod 50. In a state in which the second forceps piece 53 is in the open state with respect to the rod 50, the surgeon disposes the second end portion Tb between the second forceps piece 53 and the rod 50.

### <Lifting Step>

Here, the second end portion Tb of the defective part G is disposed at a position, at which it can be likely to be observed, with the image capturing unit of the endoscope 200 sandwiched therebetween. For this reason, it is easy for the surgeon to be able to dispose the distal end portion 501 of the rod 50 on the abdominal cavity side P1 of the muscle layer M2 exposed to the second end portion Tb. In a state in which the first end portion Ta is gripped by the rod 50 and the first forceps piece 52, the surgeon can lift up the muscle layer M2 of the second end portion Tb of the defective part G from the abdominal cavity side P1 to the stomach inward side P2 using the rod 50. Similar to the first end portion Ta, the second end portion Tb of the defective part G may be pulled from the abdominal cavity side P1 to the stomach inward side P2 using the second convex portion 501b of the distal end portion 501 of the rod 50. In the second end portion Tb of the defective part G, both the muscle layer M2 and the mucous membrane layer Ml are gripped by the second forceps piece 53 and the rod 50.

### <Second Gripping Step>

FIG. 27 is a view showing a second gripping step of the gripping forceps 400.

If the surgeon can confirm that the second end portion Tb is disposed between the second forceps piece 53 and the rod 50, the second slider 83 of the forceps manipulation portion 8 is caused to slide to the proximal end side A2 in the longitudinal direction A in the forceps manipulation portion main body 81. Consequently, the second manipulation wire 72 fixed to the second slider 83 moves to the proximal end side A2 in the longitudinal direction A inside the forceps sheath 6. The distal end portion of the second manipulation wire 72 rotates the second coupling portion 532 so as to be drawn to the groove portions 503h provided in the coupling portion 503. Consequently, the second forceps piece 53 turns to the downward side D2 with the coupling pin 54 as a rotation center. The second forceps piece 53 turns to the downward side D2 in the opening/closing direction D so as to approach the distal end side A1 of the rod 50. As a result, the second forceps piece 53 is in the closed state with respect to the rod 50, and the rod 50 and the second forceps piece 53 grip the second end portion Tb of the defective part G.

Similar to the first gripping step, the surgeon may execute the regrasping step after the second gripping step has been executed. Through this regrasping step, the surgeon can execute the first gripping step and the second gripping step over and over again. In addition, the surgeon may rotate the forceps gripping portion 5 in the midst of the second gripping step.

### <Simultaneous Opening/Closing Step>

In addition, the surgeon may simultaneously execute the first opening/closing step and the second opening/closing step. The surgeon moves the simultaneous opening/closing assistance portion (abutment portion) 84 provided in the forceps manipulation portion 8 to the distal end side A1 in the longitudinal direction A. The simultaneous opening/closing assistance portion 84 can simultaneously abut the proximal end portion of the first slider 82 and the proximal end portion of the second slider 83 and cause the first slider 82 and the second slider 83 to simultaneously slide to the distal end side A1 in the longitudinal direction A. With this constitution, the simultaneous opening/closing assistance portion 84 can simultaneously open the first forceps piece 52 and the second forceps piece 53 with the first slider 82 and the second slider 83 sandwiched therebetween.

### <Drawing Step>

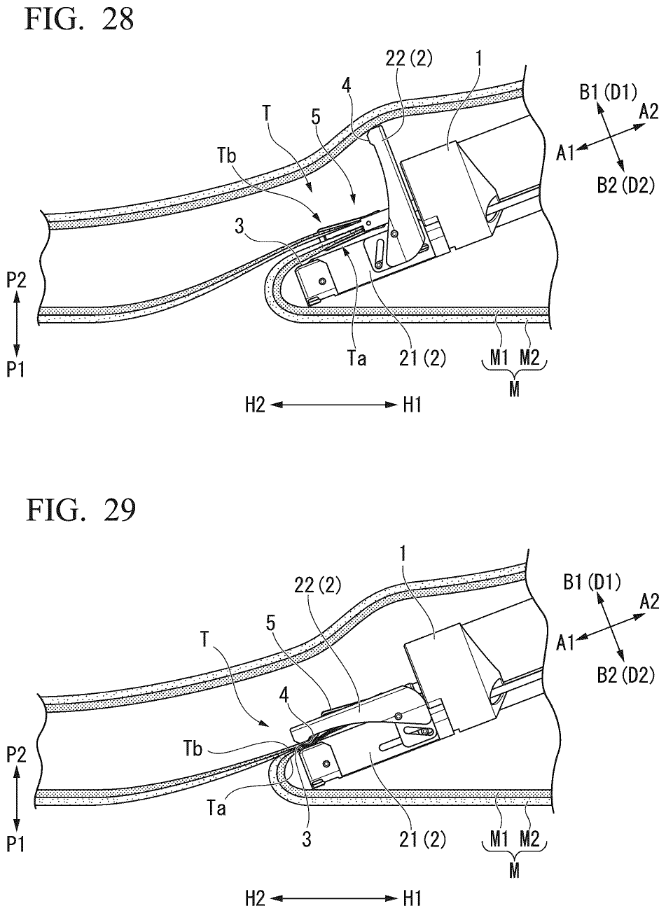

FIG. 28 is a view showing a drawing step.

The surgeon holds the finger hooking portion 86 of the forceps manipulation portion 8 and hauls the gripping forceps 400 to the proximal end side. The first forceps piece 52 and the second forceps piece 53 are drawn to the proximal end side in a state in which the first end portion Ta and the second end portion Tb of the defective part (resection hole) G are gripped. The surgeon causes the gripping forceps 400 to retract such that the distal ends of the gripping forceps 400 are disposed on the proximal end side from the staple discharge portion 3.

### <Suturing Step>

FIGS. 29 to 31 are views showing a suturing step.

When the surgeon causes the opening/closing manipulation wire 254 to retract by manipulating the opening/closing manipulation portion 250, the stapler gripping portion 2 is in the closed state. The surgeon pinches a suturing spot positioned on the outward side with respect to the defective part G from the first end portion Ta and the second end portion Tb in peripheral tissues using the staple discharge portion 3 of the first stapler gripping member 21 and the staple accommodation portion 4 of the second stapler gripping member 22. The suturing spot may be subjected to marking or the like such that the surgeon can visually recognize it.

When the stapler gripping portion 2 is in the closed state, as shown in FIG. 29, the optical axis A10 of the objective lens 215 passes through the upward side B1 of the first stapler gripping member 21 and the second stapler gripping member 22. For this reason, the surgeon can observe the treatment target T with the image capturing unit of the endoscope 200 sandwiched therebetween even when the stapler gripping portion 2 is in the closed state.

In a state in which the suturing spot is pinched by the staple discharge portion 3 and the staple accommodation portion 4, the surgeon ejects the stored staples S toward the staple accommodation portion 4 by manipulating the discharge manipulation portion 270 to drag the discharge manipulation wire 274. The needle tips S1 of the staples S penetrate the suturing spot and come into contact with the pockets 41 of the staple accommodation portion 4, thereby being bent. As a result, the suturing spot is sutured.

When suturing of the defective part G is insufficient, the surgeon may perform suturing at suturing spots of other parts. If all suturing ends, the surgeon completes suturing treatment using the gripping forceps 400 with respect to the defective part G which has been formed after full-thickness resection.

According to the suturing method of the present embodiment, when full-thickness resection is performed on the digestive tract such as the stomach, even when air inside the digestive tract escapes to the abdominal cavity side P1, causing the digestive tract to collapse and become deformed, the surgeon can accurately grip the stomach wall M of the stomach and execute suturing treatment using the gripping forceps 400.

In addition, in the present embodiment, the first forceps piece 52 and the second forceps piece 53 of the forceps pieces 51 provided in the forceps gripping portion 5 of the gripping forceps 400 can be opened and closed independently. For this reason, it is easy for the surgeon to be able to perform suturing by gripping the defective part G and drawing a tissue to the proximal end side A2 of the gripping forceps 400 using the gripping forceps 400.

In addition, in the present embodiment, the rod 50 provided in the forceps gripping portion 5 of the gripping forceps 400 includes the distal end portion 501. For this reason, the surgeon can thrust the distal end portion 501 into the defective part G and hook it onto the circumferential edge.

In addition, in the present embodiment, the distal end portion 501 includes the first convex portion 501a which protrudes to the downward side D2 and is longer than the protrusion length of the second convex portion 501b protruding to the upward side D1 in the vertical direction D. For this reason, it is easy for the surgeon to be able to hook the first convex portion 501a from the abdominal cavity side P1 so as to be locked in the muscle layer M2 of the first end portion Ta even if the first end portion Ta of the defective part G on the near side Hl cannot be accurately visually recognized. Furthermore, in a state in which the first convex portion 501a is hooked and locked in the muscle layer M2, the surgeon can pull the first end portion Ta entirely with the muscle layer M2 to the stomach inward side P2 opposite to the abdominal cavity side P1.

In addition, in the present embodiment, the central axis O4 of the rod-shaped portion 502 in the longitudinal direction A provided in the rod 50 is eccentric to the upward side D1 that is the second protrusion side from the central axis O3 of the forceps sheath 6. For this reason, even if the protrusion length of the first convex portion 501a is longer than the protrusion length of the second convex portion 501b, the gripping forceps 400 can be produced without changing the overall length and size in the opening/closing direction D.

In addition, in the present embodiment, the rod 50 includes the first projection portions 502a and the second projection portions 502b in the rod-shaped portion 502. For this reason, when the rod 50 grips a living body tissue with the forceps pieces 51, the living body tissue can be hooked by the first projection portions 502a and the second projection portions 502b. For this reason, in the gripping forceps 400, a tissue gripped by the forceps pieces 51 and the rod 50 can be unlikely to slip off.

In addition, in the present embodiment, while a state in which the first end portion Ta of the defective part G is gripped by the first forceps piece 52 and the rod 50 is maintained, the surgeon moves the gripping forceps 400 and the endoscope 200 in their entirety to a location near the second end portion Tb on the far side H2. For this reason, the surgeon can move the forceps gripping portion 5 in order to easily grip the far side H2 in a state in which both the muscle layer M2 and the mucous membrane layer M1 of the first end portion Ta are gripped even if the defective part G cannot be accurately visually recognized.

Hereinabove, the first embodiment of the present invention has been described in detail with reference to the drawings. However, specific constitutions are not limited to this embodiment, and design change and the like within a range not departing from the gist of the present invention are also included. In addition, the constituent elements in the embodiment and the modification example described above can be constituted in a suitable combination.

### Second Embodiment

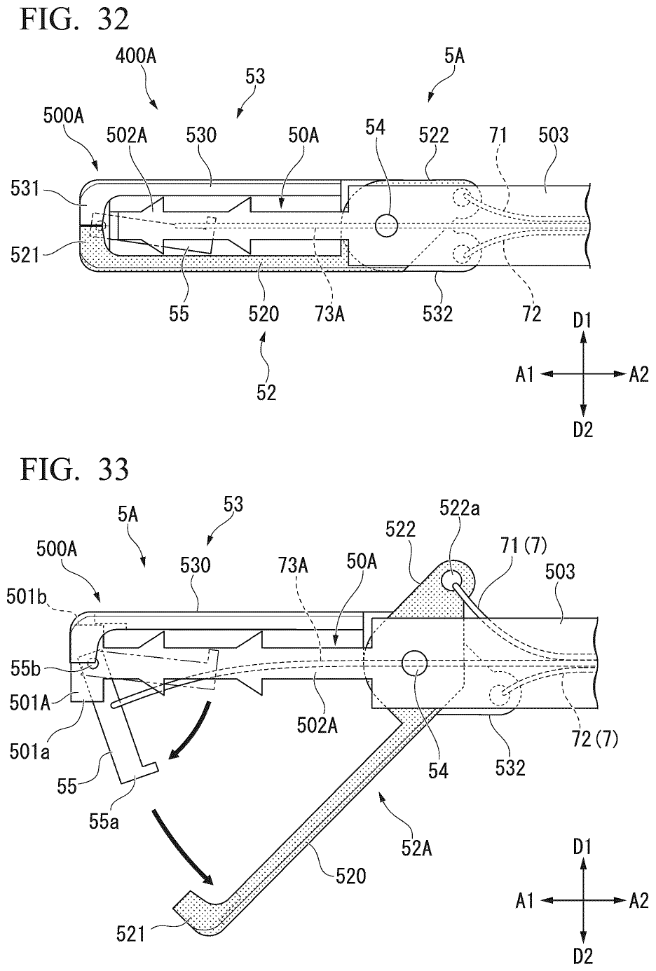

Next, a second embodiment of the present invention will be described with reference to FIGS. 32 and 33. In the following description, the same reference signs are applied to common constituents which have already been described, and duplicate description will be omitted. In all the following embodiments, compared to the first embodiment, gripping forceps attached to the endoscope differ. Therefore, the following description will focus on points different from those in the first embodiment.

FIG. 32 is an enlarged view of the distal end side A1 of gripping forceps. FIG. 33 is a side view showing a state in which a first forceps piece 52A of the gripping forceps is in the open state and the second forceps piece 53 is in the closed state.

A forceps gripping portion 5A of the gripping forceps (endoscopic treatment tool) according to the second embodiment of the present invention includes a rod 50A, the forceps piece (jaw) 51, and the coupling pin 54, as shown in FIG. 32. In addition, the rod 50A has a distal end portion 501A (refer to FIG. 33) and the rod-shaped portion 502A of a loading portion 500A, and the coupling portion 503. The forceps pieces 51 include the first forceps piece 52A and the second forceps piece 53.

As shown in FIG. 33, compared to the first embodiment, the distal end portion 501A is formed symmetrically with respect to the central axis of a rod-shaped portion 502A in the longitudinal direction A. In addition, as shown in FIG. 33, the distal end portion 501A includes an anchor (first convex portion) 55 extending to a downward side (first protrusion side) B2 in a vertical direction (protrusion direction) B from the distal end portion 501A when the first forceps piece 52A of the forceps pieces 51 is in the open state with respect to the rod 50A. Similar to the first embodiment, the distal end portion 501A may be formed non-symmetrically with respect to the central axis of the rod-shaped portion 502A.

The anchor (first convex portion) 55 is a rod-shaped member in which a fixed end of the anchor 55 is coupled to a central portion of the distal end portion 501A. As shown in FIG. 33, the anchor 55 includes a fixing pin (fixing portion) 55b at the fixed end. The anchor 55 can turn about the fixing pin 55b on the proximal end side A2 in the longitudinal direction A from the distal end portion 501A. In addition, the anchor 55 further includes a convex portion wire 73A.

One end of the convex portion wire 73A is coupled to a movable end side on a side opposite to the fixed end of the anchor 55 from the fixing pin 55b. The other end of the convex portion wire 73A is connected to a first manipulation wire (first wire) 71 coupled to the first coupling portion 522 of the first forceps piece 52A. If the first manipulation wire 71 advances and retracts in the longitudinal direction A, the convex portion wire 73A advances and retracts in the longitudinal direction A in association with the first manipulation wire 71.

As shown in FIG. 33, if the convex portion wire 73A advances to the distal end side A1 in the longitudinal direction A in association with the first manipulation wire 71, the anchor 55 turns about the fixing pin 55b, and a distal end portion 55a protrudes to the downward side D2 from the distal end portion 501A. When the first forceps piece 52A of the forceps pieces 51 is in the open state with respect to the rod 50A, the anchor 55 is in a state of protruding toward the first forceps piece 52A provided on the downward side D2 in the vertical direction D from the distal end portion 501A. At this time, the distal end portion 501A of the rod 50A includes the anchor (first convex portion) 55 which is longer than the protrusion length of the second convex portion 501b protruding to the upward side D1 in the vertical direction D and protrudes to the downward side D2.

In addition, as shown in FIG. 32, if the convex portion wire 73A moves to the proximal end side A2 in the longitudinal direction A in association with the first manipulation wire 71, the anchor 55 turns such that the distal end portion 55a is drawn to the proximal end side A2. When the first forceps piece 52A of the forceps pieces 51 is in the closed state with respect to the rod 50A, the anchor 55 is in a folded state between the first forceps piece 52A and the rod 50A with the distal end portion 55a facing the proximal end side A2.

Compared to the first forceps piece 52 of the first embodiment, the first forceps piece 52A has a marker which is visually distinguishable from the second forceps piece 53. In the present embodiment, the first forceps piece 52A has a color different from that of the second forceps piece 53. The first forceps piece 52A need only be visually distinguishable from the second forceps piece 53 and may have a pattern or a different shape, for example.

In the present embodiment, similar to the first embodiment described above, since the distal end portion 501A of the rod 50A includes the anchor (first convex portion) 55, even if the first end portion Ta of the defective part G on the near side H1 cannot be accurately visually recognized, the surgeon can hook and lock the anchor 55 from the abdominal cavity side P1 of the muscle layer of the first end portion Ta and pull it entirely with the muscle layer M2 toward the stomach inward side P2.

In addition, in the present embodiment, when the first forceps piece 52A of the forceps pieces 51 is in the closed state with respect to the rod 50A, the anchor 55 is folded between the first forceps piece 52A and the rod 50A. For this reason, when the first forceps piece 52A is closed with respect to the rod 50A, it does not interfere with the rod 50A.

In addition, in the present embodiment, the first forceps piece 52A has a marker which is visually distinguishable from the second forceps piece 53. For this reason, when the surgeon manipulates the gripping forceps with the image capturing unit of the endoscope 200 sandwiched therebetween, it is easy to be able to confirm the first forceps piece 52A which is being opened and closed on a downward side DI where the anchor 55 is disposed with respect to the rod 50A.

Hereinabove, the second embodiment of the present invention has been described in detail with reference to the drawings. However, specific constitutions are not limited to this embodiment, and design change and the like within a range not departing from the gist of the present invention are also included. In addition, the constituent elements described in the foregoing embodiments and the following modification examples can be constituted in a suitable combination.

### Third Embodiment

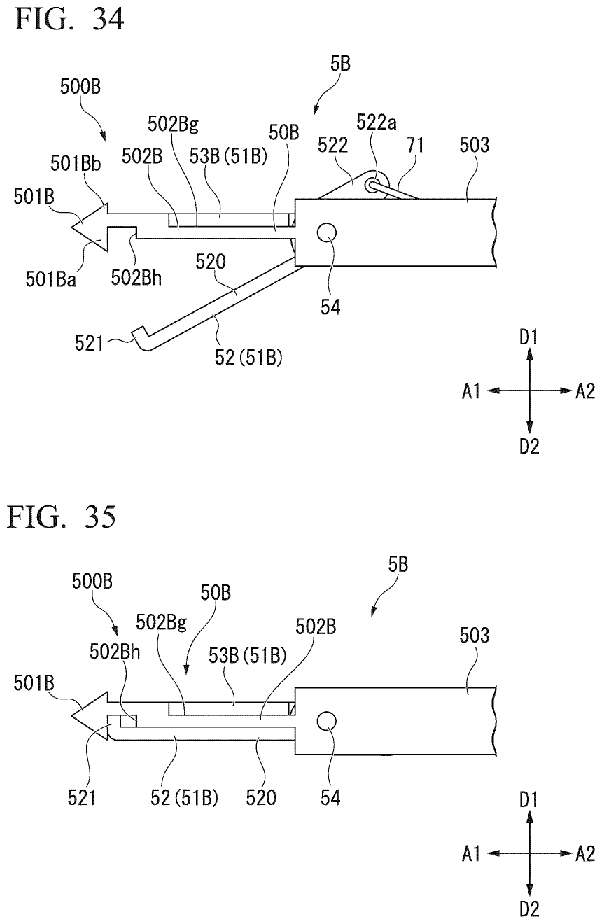

Next, a third embodiment of the present invention will be described with reference to FIGS. 34 and 35. In the present embodiment, compared to the first embodiment, the forceps gripping portion of the gripping forceps differs. Therefore, the following description will focus on points different from those in the first embodiment.

FIG. 34 is an enlarged view of the distal end side A1 of gripping forceps. FIG. 35 is a side view showing a state in which the first forceps piece 52 of the gripping forceps is in the closed state and a second forceps piece 53B is in the closed state.

A forceps gripping portion 5B of the gripping forceps (endoscopic treatment tool) according to the third embodiment of the present invention includes a rod 50B, a forceps piece (jaw) 51B, and the coupling pin 54 as shown in FIG. 34. In addition, the rod 50B has a distal end portion 501B and a rod-shaped portion 502B of a loading portion 500B, and the coupling portion 503.

As shown in FIGS. 34 and 35, compared to the first embodiment, the distal end portion 501B is formed to have a substantially triangular shape so as to be tapered to the distal end side A1 in the longitudinal direction A. In addition, the distal end portion 501B includes a second convex portion 501Bb protruding to the upward side D1 in the vertical direction D, and a first convex portion 501Ba longer than the protrusion length of the second convex portion 501Bb and protruding to the downward side D2 in the vertical direction D.

The rod-shaped portion 502B includes a first concave portion 502Bh recessed to the upward side D1 in the vertical direction D on the distal end side A1 in the longitudinal direction A. The depth of the first concave portion 502Bh in the vertical direction D is substantially the same as the length of the engagement portion 521 of the first forceps piece 52. For this reason, in the engagement portion 521 of the first forceps piece 52, as shown in FIG. 35, when the first forceps piece 52 of the forceps pieces 51B is in the closed state with respect to the rod 50B, the engagement portion 521 is accommodated in the first concave portion 502Bh. In addition, on the proximal end side A2 from the first concave portion 502Bh, the rod-shaped portion 502B includes a second concave portion 502Bg recessed in the longitudinal direction on the downward side D2 in the vertical direction D. The second concave portion 502Bg is formed by hollowing out a concave portion in substantially the same shape as the second forceps piece 53B of the forceps pieces 51B. The rod-shaped portion 502B may further include the first projection portions 502a and the second projection portions 502b.

The second forceps piece 53B of the forceps pieces 51B has a length in the longitudinal direction A shorter than the length of the first forceps piece 52. As shown in FIG. 35, when the second forceps piece 53B is in the closed state with respect to the rod 50B, it is accommodated in the second concave portion 502Bg provided in the rod-shaped portion 502B.

In the present embodiment, when the first forceps piece 52 of the forceps pieces 51B is in the closed state with respect to the rod 50B, the engagement portion 521 is accommodated in the first concave portion 502Bh. In addition, when the second forceps piece 53B is in the closed state with respect to the rod 50A, it is accommodated in the second concave portion 502Bg provided in the rod-shaped portion 502B. For this reason, the widths and the thicknesses of the gripping forceps in the vertical direction D and the width direction E can be made as small as possible, and suturing treatment with respect to the small defective part (resection hole) G can also be facilitated easily.

Hereinabove, the third embodiment of the present invention has been described in detail with reference to the drawings. However, specific constitutions are not limited to this embodiment, and design change and the like within a range not departing from the gist of the present invention are also included. In addition, the constituent elements described in the foregoing embodiments and the following modification examples can be constituted in a suitable combination.

### (Modification Example)

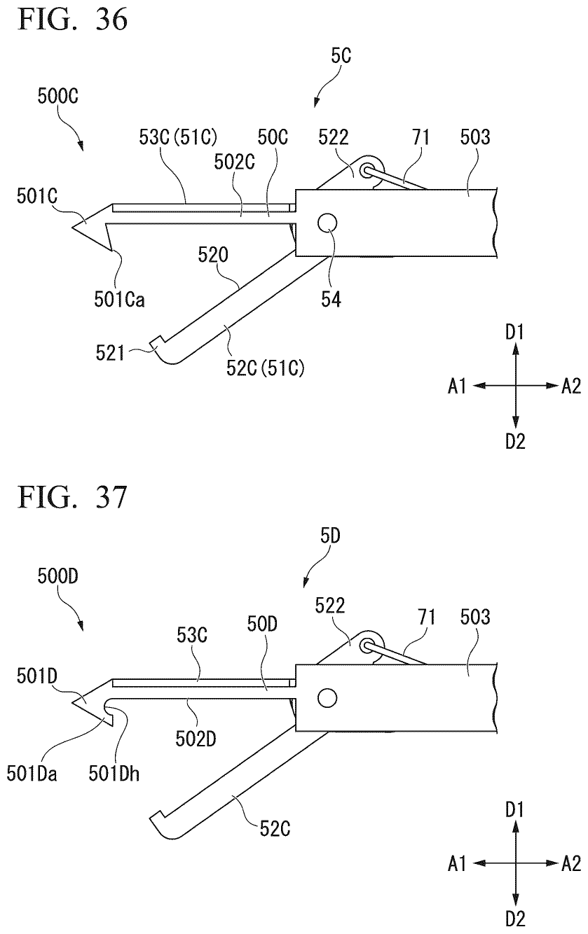

In the distal end portion provided in the rod of the gripping forceps according to the embodiments described above, the distal end may be formed toward the proximal end side in the longitudinal direction. For example, as shown in FIG. 36, a rod 50C of a forceps gripping portion 5C includes a distal end portion 501C provided in a loading portion 500C. Compared to the first convex portion 501Ba of the third embodiment, a first convex portion 501Ca of the distal end portion 501C is formed with the distal end facing the proximal end side A2 in the longitudinal direction A. With this constitution, the first convex portion 501Ca of the distal end portion 501C is more likely to be hooked onto a living body tissue.

In addition, the thickness of the forceps piece of the gripping forceps according to the embodiments described above is not particularly limited. For example, as shown in FIG. 36, the thickness of a second forceps piece 53C in a transverse direction perpendicular to the longitudinal direction A is formed to be smaller than that of a first forceps piece 52C. With this constitution, in the gripping forceps, when the forceps piece is in the closed state with respect to the rod, the widths and the thicknesses of the gripping forceps in the vertical direction D and the width direction E can be formed to be smaller.

In addition, as shown in FIG. 37, a rod 50D of the gripping forceps according to the embodiments described above may include a concave portion 501Dh recessed toward the distal end side A1 in the longitudinal direction A on a surface (hooking surface) provided on the proximal end side A2 in the longitudinal direction A in a first convex portion 501Da of a distal end portion 501D provided in a loading portion 500D. With this constitution, after the first convex portion 501Da is hooked onto the muscle layer of the first end portion Ta, a tissue can enter the concave portion 501Dh so that it can be unlikely to come off from the first convex portion 501Da.

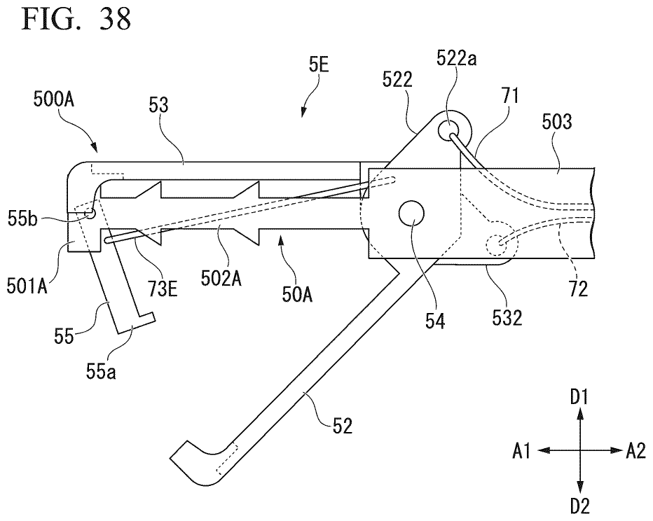

In addition, in a convex portion wire 73E of a rod 50A of the gripping forceps according to the embodiments described above, as shown in FIG. 38, compared to the convex portion wire 73A of the second embodiment, the other end may be connected to the first coupling portion 522 of the first forceps piece 52. With this constitution as well, the first coupling portion 522 turns if the first manipulation wire 71 advances and retracts in the longitudinal direction A so that the convex portion wire 73E can advance and retract in the longitudinal direction A in association with the first coupling portion 522.

In addition, the anchor 55 of the gripping forceps according to the second embodiment does not have to have the convex portion wire 73A by being given elasticity when it moves to the upward side from a state of protruding to the downward side. In this case as well, the surgeon can pull the entire muscle layer toward the stomach inward side by hooking and locking the anchor 55 from the abdominal cavity side of the muscle layer. In addition, since the anchor 55 is thrust to the upward side when the first forceps piece of the forceps pieces is displaced from the open state to the closed state with respect to the rod, the anchor 55 can be folded between the first forceps piece and the rod.

In all the foregoing embodiments, according to the gripping forceps and the suturing method of the present invention, a mucous membrane layer and a muscle layer at a circumferential edge of a resection hole can be reliably gripped, and the resection hole can be sutured using the gripping forceps.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. Gripping forceps comprising:
a sheath which extends in a longitudinal direction; and
a gripping portion which has a rod provided at a distal end of the sheath and extending in the longitudinal direction, a first jaw and a second jaw are configured to open or close toward the rod,
wherein the rod has a distal end portion which includes a first convex portion protruding to a first protrusion side toward the first jaw.

2. The gripping forceps according to claim 1,
wherein a central axis of the rod in the longitudinal direction is eccentric to a side opposite to the first protrusion side from a central axis of the sheath in the longitudinal direction.

3. The gripping forceps according to claim 1,
wherein the first convex portion has a fixing portion which is provided on a fixed end side of the first convex portion and coupled to the rod, and is turnable to a proximal end side in the longitudinal direction about the fixing portion.

4. The gripping forceps according to claim 3 further comprising:
a convex portion wire in which one end is coupled to a movable end side on a side opposite to the fixed end side from the fixing portion and the other end is connected to the first jaw in the first convex portion,
wherein the convex portion wire interlocks the first convex portion with the first jaw and turns the first convex portion.

5. The gripping forceps according to claim 3 further comprising:
a first wire which is coupled so as to allow the first jaw to be opened and closed and advances and retracts in the longitudinal direction; and
a convex portion wire in which one end is coupled to a movable end side on a side opposite to the fixed end side from the fixing portion and the other end is connected to the first wire in the first convex portion,
wherein the convex portion wire interlocks the first convex portion with the first wire and turns the first convex portion.

6. Gripping forceps comprising:
a sheath which extends in a longitudinal direction; and
a gripping portion which has a rod provided at a distal end of the sheath and extending in the longitudinal direction, a first jaw coupled to a proximal end side of the rod and being opened and closed toward a distal end side of the rod, and a second jaw coupled to the proximal end side of the rod on a side opposite to the first jaw with the rod sandwiched therebetween and being opened and closed toward the distal end side of the rod,
wherein the rod has a first convex portion which protrudes to a first protrusion side toward the first jaw on the distal end side, and
a central axis of the rod in the longitudinal direction is eccentric to a second protrusion side on a side opposite to the first protrusion side from a central axis of the sheath in the longitudinal direction.

7. The gripping forceps according to claim 6,
wherein the first jaw has a marker which is visually distinguishable from the second jaw.

8. The gripping forceps according to claim 6,
wherein the first convex portion has a fixing portion which is provided on a fixed end side of the first convex portion and coupled to the rod, and is turnable to the proximal end side in the longitudinal direction about the fixing portion.

9. The gripping forceps according to claim 8 further comprising:
a convex portion wire in which one end is coupled to a movable end side on a side opposite to the fixed end side from the fixing portion and the other end is connected to the first jaw in the first convex portion,
wherein the convex portion wire interlocks the first convex portion with the first jaw and turns the first convex portion.

10. The gripping forceps according to claim 8 further comprising:
a first wire which is coupled so as to allow the first jaw to be opened and closed and advances and retracts in the longitudinal direction; and
a convex portion wire in which one end is coupled to a movable end side on a side opposite to the fixed end side from the fixing portion and the other end is connected to the first wire in the first convex portion,
wherein the convex portion wire interlocks the first convex portion with the first wire and turns the first convex portion.

11. A suturing method in which a tissue having a resection hole is gripped and sutured using gripping forceps, the suturing method comprising:
a thrusting step of thrusting a rod, which is provided in the gripping forceps and disposed in a tangent direction of the resection hole, into the resection hole;
a pulling step of pulling a first end portion, which is an end portion on a near side in the tangent direction, from a muscle layer side of the tissue using the rod at a circumferential edge provided in the resection hole;
a first gripping step of gripping the first end portion using the rod and a first jaw which is provided on a distal end side of the gripping forceps, coupled to the rod, and opened and closed toward a distal end side of the rod;
a dragging step of causing the rod and the first jaw to advance to a location near a second end portion which is an end portion on a far side in the tangent direction at the circumferential edge in a state in which the first end portion is gripped;
a second gripping step of gripping the second end portion using the rod and a second jaw which is provided on the distal end side of the gripping forceps, coupled to the rod on a side opposite to the first jaw with the rod sandwiched therebetween, and opened and closed toward the distal end side of the rod; and
a drawing step of drawing the gripping forceps to a proximal end side in a state in which the first end portion and the second end portion are gripped.

12. The suturing method according to claim 11 further comprising:
a lifting step of lifting up the second end portion using the rod from the muscle layer side in the tissue in a state in which the first end portion is gripped.

13. The suturing method according to claim 11 further comprising:
a rotating step of rotating the gripping forceps about a central axis in a longitudinal direction to dispose the first jaw on the muscle layer side.

14. The suturing method according to claim 11 further comprising:
a hooking step of hooking a first convex portion, which protrudes to a first protrusion side in a protrusion direction toward the first jaw provided on the distal end side of the rod, in the muscle layer of the tissue.

15. The suturing method according to claim 11 further comprising:
a suturing step of suturing the first end portion and the second end portion in a state in which the first end portion and the second end portion are drawn to a proximal end side of the gripping forceps.
